(19) United States
(12) Patent Application Publication
(10) Pub. No.: US 2018/0296855 A1
(43) Pub. Date: Oct. 18, 2018

(54) BRACHYTHERAPY APPLICATOR SYSTEMS AND METHODS

(71) Applicant: Salutaris Medical Devices, Inc., Tucson, AZ (US)

(72) Inventors: Ryan L. Lohrenz, Vail, AZ (US); Laurence J. Marsteller, Tucson, AZ (US); Tianna D. Benson, Tucson, AZ (US); Samuel S. Hyman, Tucson, AZ (US); Mary E. Drew, Oro Valley, AZ (US); Russell J. Hamilton, Tucson, AZ (US); Wendell Lutz, Tucson, AZ (US)

(21) Appl. No.: 16/015,892

(22) Filed: Jun. 22, 2018

Related U.S. Application Data
(63) Continuation-in-part of application No. 15/004,538, filed on Jan. 22, 2016, now Pat. No. 10,022,558.
Application No. 15/004,538 is a continuation-in-part of application No. 13/953,528, filed on Jul. 29, 2013, now abandoned.
Application No. 15/004,538 is a continuation-in-part of application No. 13/872,941, filed on Apr. 29, 2013, now abandoned.
Application No. 13/872,941 is a division of application No. 12/350,079, filed on Jan. 7, 2009, now Pat. No. 8,430,804.
Application No. 15/004,538 is a continuation-in-part of application No. 14/486,401, filed on Sep. 15, 2014, now Pat. No. 9,873,001.
Application No. 14/486,401 is a continuation of application No. 13/872,941, filed on Apr. 29, 2013, now abandoned.
Application No. 13/872,941 is a division of application No. 12/350,079, filed on Jan. 7, 2009, now Pat. No. 8,430,804.
Application No. 14/486,401 is a continuation-in-part of application No. 13/953,528, filed on Jul. 29, 2013, now abandoned.
Application No. 14/486,401 is a continuation-in-part of application No. 14/011,516, filed on Aug. 27, 2013, now Pat. No. 9,056,201.
Application No. 14/011,516 is a continuation-in-part of application No. 13/742,823, filed on Jan. 16, 2013, now Pat. No. 8,597,169.
Application No. 13/742,823 is a continuation-in-part of application No. 12/497,644, filed on Jul. 3, 2009, now abandoned.
Application No. 14/011,516 is a continuation-in-part of application No. 13/872,941, filed on Apr. 29, 2013, now abandoned.
Application No. 13/872,941 is a division of application No. 12/350,079, filed on Jan. 7, 2009, now Pat. No. 8,430,804.
Application No. 14/011,516 is a continuation-in-part of application No. 13/111,780, filed on May 19, 2011, now Pat. No. 8,608,632.
Application No. 13/111,780 is a continuation-in-part of application No. 12/497,644, filed on Jul. 3, 2009, now abandoned.
Application No. 12/497,644 is a continuation-in-part of application No. 12/350,079, filed on Jan. 7, 2009, now Pat. No. 8,430,804.
Application No. 14/011,516 is a continuation-in-part of application No. 12/917,044, filed on Nov. 1, 2010, now abandoned.
Application No. 14/011,516 is a continuation-in-part of application No. 13/111,765, filed on May 19, 2011, now Pat. No. 8,602,959.
Application No. 14/011,516 is a continuation-in-part of application No. 13/953,528, filed on Jul. 29, 2013, now abandoned.
Application No. 13/953,528 is a continuation-in-part of application No. PCT/US16/68391, filed on Dec. 22, 2016.
(60) Provisional application No. 61/676,783, filed on Jul. 27, 2012.
(60) Provisional application No. 61/877,765, filed on Sep. 13, 2013.
(60) Provisional application No. 61/676,783, filed on Jul. 27, 2012.
(60) Provisional application No. 61/347,226, filed on May 21, 2010.
(60) Provisional application No. 61/257,232, filed on Nov. 2, 2009.
(60) Provisional application No. 61/376,115, filed on Aug. 23, 2010.
(60) Provisional application No. 61/347,233, filed on May 21, 2010.
(60) Provisional application No. 61/676,783, filed on Jul. 27, 2012.
(60) Provisional application No. 61/047,693, filed on Apr. 24, 2008.
(60) Provisional application No. 61/035,371, filed on Mar. 10, 2008.
(60) Provisional application No. 61/033,238, filed on Mar. 3, 2008.
(60) Provisional application No. 61/010,322, filed on Jan. 7, 2008.
(60) Provisional application No. 62/334,876, filed on May 11, 2016.
(60) Provisional application No. 62/271,169, filed on Dec. 22, 2015.

(51) Int. Cl. A61N 5/10

(52) U.S. Cl. CPC A61N 5/1017; A61M 25/01; A61M 2210/0612

(57) ABSTRACT

Brachytherapy applicator systems and methods of use such as methods of introducing radiation to a target using the brachytherapy applicator systems. The systems may feature a cannula attached to a handle, wherein the cannula has a distal portion connected to a proximal portion. A holder may be disposed at the tip of the distal portion for holding a radionuclide brachytherapy source (RBS). The RBS may be pre-loaded or loaded into the cannula at the time of treatment (or at an appropriate time prior to treatment).

### CROSS REFERENCE

This application is a continuation in-part and claims benefit of PCT Patent Application No. PCT/US2016/068391 filed Dec. 22, 2016, which claims benefit of U.S. Provisional Application No. 62/334,876 filed May 11, 2016 and U.S. Provisional Application No. 62/271,169 filed Dec. 22, 2015, the specification(s) of which is/are incorporated herein in their entirety by reference.

This application is a continuation in-part and claims benefit of U.S. patent application Ser. No. 15/004,538 filed Jan. 22, 2016, the specification(s) of which is/are incorporated herein in their entirety by reference.

U.S. patent application Ser. No. 15/004,538 is a continuation in-part and claims benefit of U.S. patent application Ser. No. 13/953,528 filed Jul. 29, 2013, which is a non-provisional of U.S. Provisional Application No. 61/676,783 filed Jul. 27, 2012, the specification(s) of which is/are incorporated herein in their entirety by reference.

U.S. patent application Ser. No. 15/004,538 is also a continuation in-part and claims benefit of U.S. patent application Ser. No. 13/872,941 filed Apr. 29, 2013, which is a divisional of U.S. patent application Ser. No. 12/350,079 filed Jan. 7, 2009 and now U.S. Pat. No. 8,430,804.

U.S. patent application Ser. No. 15/004,538 is also a continuation in-part and claims benefit of U.S. patent application Ser. No. 14/486,401 filed on Sep. 15, 2014 and now U.S. Pat. No. 9,873,001, which is a non-provisional of U.S. Provisional Patent Application No. 61/877,765 filed Sep. 13, 2013, the specification(s) of which is/are incorporated herein in their entirety by reference.

U.S. patent application Ser. No. 14/486,401 is also a continuation in-part of U.S. patent application Ser. No. 13/872,941 filed Apr. 29, 2013, which is a divisional of U.S. patent application Ser. No. 12/350,079 filed Jan. 7, 2009 and now U.S. Pat. No. 8,430,804.

U.S. patent application Ser. No. 14/486,401 is also a continuation in-part of U.S. patent application Ser. No. 13/953,528 filed Jul. 29, 2013, which is a non-provisional of U.S. Provisional Application No. 61/676,783 filed Jul. 27, 2012, the specification(s) of which is/are incorporated herein in their entirety by reference.

U.S. patent application Ser. No. 14/486,401 is also a continuation in-part of U.S. patent application Ser. No. 14/011,516 filed Aug. 27, 2013 and now U.S. Pat. No. 9,056,201, which is a continuation in-part of U.S. patent application Ser. No. 13/742,823 filed Jan. 16, 2013 and now U.S. Pat. Bo. 8,597,169, which is a continuation of U.S. patent application Ser. No. 12/497,644 filed Jul. 3, 2009, which is a continuation-in-part of U.S. patent application Ser. No. 12/350,079 filed Jan. 7, 2009 and now U.S. Pat. No. 8,430,804.

U.S. patent application Ser. No. 14/011,516 is also a continuation in-part of U.S. patent application Ser. No. 13/872,941 filed Apr. 29, 2013, which is a divisional of U.S. patent application Ser. No. 12/350,079 filed Jan. 7, 2009 and now U.S. Pat. No. 8,430,804.

U.S. patent application Ser. No. 14/011,516 is also a continuation in-part of U.S. patent application Ser. No. 13/111,780 filed May 19, 2011 and now U.S. Pat. No. 8,608,632, which is a non-provisional of U.S. Provisional Application No. 61/347,226 filed May 21, 2010; and a continuation-in-part of U.S. patent application Ser. No. 12/497,644 filed Jul. 3, 2009, which is a continuation-in-part of U.S. patent application Ser. No. 12/350,079 filed Jan. 7, 2009 and now U.S. Pat. No. 8,430,804.

U.S. patent application Ser. No. 14/011,516 is also a continuation in-part of U.S. patent application Ser. No. 12/917,044 filed Nov. 1, 2010, which is a non-provisional of U.S. Provisional Application No. 61/257,232 filed Nov. 2, 2009 and U.S. Provisional Application No. 61/376,115 filed Aug. 23, 2010, the specification(s) of which is/are incorporated herein in their entirety by reference.

U.S. patent application Ser. No. 14/011,516 is also a continuation in-part of U.S. patent application Ser. No. 13/111,765 filed May 19, 2011 and now U.S. Pat. No. 8,602,959, which is a non-provisional of U.S. Provisional Application No. 61/347,233 filed May 21, 2010, the specification(s) of which is/are incorporated herein in their entirety by reference.

U.S. patent application Ser. No. 14/011,516 is also a continuation in-part of U.S. patent application Ser. No. 13/953,528 filed Jul. 29, 2013, which is a non-provisional of U.S. Provisional Application No. 61/676,783 filed Jul. 27, 2012, the specification(s) of which is/are incorporated herein in their entirety by reference.

U.S. patent application Ser. No. 12/350,079, now U.S. Pat. No. 8,430,804, is a non-provisional of U.S. Provisional Application No. 61/010,322 filed Jan. 7, 2008, U.S. Provisional Application No. 61/033,238 filed Mar. 3, 2008, U.S. Provisional Application No. 61/035,371 filed Mar. 10, 2008, and U.S. Provisional Application No. 61/047,693 filed Apr. 24, 2008, the specification(s) of which is/are incorporated herein in their entirety by reference.

### FIELD OF THE INVENTION

The present invention relates to brachytherapy devices, brachytherapy systems, and methods for introducing radiation to a target area. For example, the present invention features systems and methods for introducing radiation to the eye, e.g., for treating and/or managing eye conditions including, but not limited to, macula degeneration.

### BACKGROUND OF THE INVENTION

Brachytherapy is treatment of a region by placing radioactive isotopes in, on, or near it. Both malignant and benign conditions are successfully treated with brachytherapy. Lesion location dictates treatment technique. For the treatment of tumors or tumor beds in the breast, tongue, abdomen, or muscle capsules, catheters are inserted into the tissue (interstitial application). Radiation may be delivered by inserting strands of radioactive seeds into these catheters for a predetermined amount of time. Permanent implants are also possible. For example, in the treatment of prostate cancer, radioactive seeds are placed directly into the prostate where they remain indefinitely. Restenosis of coronary arteries after stent implantation, a non-malignant condition, has been successfully treated by placing a catheter into the coronary artery, then inserting a radioactive source into the catheter and holding it there for a predetermined time in order to deliver a sufficient dose to the vessel wall. Beta emitters, such as phosphorus 32 (P-32) and strontium 90 (Sr-90), and gamma emitters, such as iridium 192 (Ir-192), have been used. The Collaborative Ocular Melanoma Study (COMS), a multicenter randomized trial sponsored by the National Eye Institute and the National Cancer Institute demonstrated the utility of brachytherapy for the treatment of ocular cancers and/or tumors. The technique employs an invasive surgical procedure to allow placement of a surface applicator (called an episcleral plaque) that is applied extraocularly by suturing it to the sclera. The gold plaque contains an inner mold into which radioactive iodine 125 (I-125) seeds are inserted. The gold plaque serves to shield the tissues external to the eye while exposing the sclera, choroid, choroidal melanoma, and overlying retina to radiation. The plaque remains fixed for a few days to one week in order to deliver approximately 85 Gy to the tumor apex.

The present invention features brachytherapy systems (e.g., brachytherapy applicators, e.g., cannulas, etc.) for introducing radiation to a target (e.g., a target of the eye) and methods of use of said brachytherapy systems. For example, in some embodiments, the methods and systems of the present invention allow for minimally-invasive delivery of radiation to the eye, e.g., the posterior portion of the eye.

Without wishing to limit the present invention to any theory or mechanism, it is believed that the devices of the present invention are advantageous over the prior art. For example, the devices of the present invention have geometric and dosimetric advantages because they may be placed at a desired location (e.g., on the eye, within layers of the eye tissue, the macula, etc.) with accuracy (e.g., sub millimeter accuracy), and the radioisotope (e.g., beta) may be used to construct the radiation source with a predominately limited range.

### SUMMARY OF THE INVENTION

The present invention provides methods and devices for minimally-invasive deliver of radiation to the eye, e.g., to the posterior portion of the eye.

The present invention also provides brachytherapy systems (e.g., cannulas) for delivering radiation to a target, e.g., a target on the eye, a target in the eye, etc. In some embodiments, the system (e.g., cannula) is for placing under the Tenon's capsule. For example, the present invention, e.g., brachytherapy system of the present invention, features a cannula (e.g., a fixed shape cannula, a partially fixed shape cannula, flexible cannula, partially flexible cannula, etc.) comprising a distal portion for placement around a portion of a globe of an eye and a proximal portion (e.g., a curved proximal portion) connected to the distal portion via a straight portion and/or an inflection point. In some embodiments, the system (e.g., cannula) further comprises a handle extending from the proximal portion. In some embodiments, the handle lies on an axis such that the axis does not intersect with the distal portion.

In some embodiments, the system comprises a radionuclide brachytherapy source (RBS) holder for holding an RBS, e.g., an RBS holder disposed on the distal portion of the cannula, e.g., at the tip of the distal portion of the cannula. In some embodiments, the system further comprises a radionuclide brachytherapy source (RBS), e.g., an RBS disposed in the RBS holder, e.g., an RBS disposed at a treatment position, an RBS disposed at a tip of the distal portion. In some embodiments, the RBS holder is connected to the distal portion, e.g., the tip of the distal portion, via a straight distal portion (e.g., the RBS holder and tip of the distal portion are connected via a straight distal portion.

In some embodiments, the system further comprises a shield for shielding (e.g., temporarily shielding) the RBS. In some embodiments, the shield attached to (e.g., removably, slidably, etc.) to the system, e.g., to part of the distal portion of the cannula or the proximal portion of the cannula.

In some embodiments, the system further comprises a light system, e.g., a light source, a light emitting component, etc. The light system may feature a light emitting component disposed at the tip of the distal portion of the cannula, e.g., on the RBS holder at the tip of the distal portion of the cannula. Various different types of light systems may be considered. For example, in some embodiments, the light system comprises a fiber optic wire connected to a light source, wherein the light from the light source travels to the end of the fiber optic wire (the end of the fiber optic wire being the light emitting component). The end of the fiber optic wire may be at the tip of the cannula, the RBS holder at the tip of the distal portion, etc. The present invention is not limited to fiber optic wires. For example, in some embodiments, the light system (and light emitting component) comprises a light emitting diode (LED) or other appropriate light system. In some embodiments, the light emitting component extends or lies in a slot in the bottom surface of the RBS holder.

The present invention also features methods and devices for applying radiation for the treatment of diseases including but not limited to wet age-related macular degeneration, and methods for irradiating a target, e.g., a target of an eye in a patient. The brachytherapy system (e.g., cannula) size is small, which allows for minimally-invasive surgery by making a small incision in the conjunctiva and inserting the cannula under Tenon's membrane along the sclera (e.g., at the limbus but not limited to the limbus, e.g., at a point posterior to the limbus, a point between the limbus and the fornix, etc.). The method may comprise inserting a system (e.g., cannula with an RBS at a treatment position, e.g., at the tip of the distal portion of the cannula, in the RBS holder at the tip of the distal portion of the cannula, etc.) into a potential space under the Tenon's capsule. The RBS is positioned over the target and the RBS irradiates the target. In some embodiments, the Tenon's capsule guides the insertion of the cannula and provides positioning support for the cannula.

In some embodiments, when the RBS (e.g., the RBS holder on the distal portion) is positioned within the vicinity of the target, the proximal portion curves away from the visual axis as to allow a user to have direct visual access in the eye.

In some embodiments, the target is a lesion associated with the retina. In some embodiments, the target is located on the vitreous side of the eye. In some embodiments, the target (e.g., lesion) is a benign growth or a malignant growth. In some embodiments, the target (e.g., lesion) is a neovascular lesion.

In some embodiments, the brachytherapy system (e.g., cannula, etc.) is disposable, or a portion thereof is disposable. In some embodiments, the radiation source (RBS) is inserted into the disposable applicator.

The brachytherapy system may be preloaded with the RBS or afterloaded. For example, in some embodiments, the RBS is loaded into the brachytherapy system (e.g., RBS holder) before the brachytherapy system (e.g., cannula) is inserted. For example, in U.S. Pat. No. 7,070,554 to White, the brachytherapy device comprises a preloaded radiation source, i.e., a radiation source affixed at the tip of the device prior to the insertion of the device into the eye. In some embodiments, the RBS is loaded into the system (e.g., the RBS holder) after the cannula is inserted.

The RBS may be constructed to provide any dose rate to the target. In some embodiments, the RBS provides a dose rate greater than 10 Gy/min, a dose rate from 0.1 to 1 Gy/min, from 1 to 10 Gy/min, from 10 to 20 Gy/min, from 20 to 30 Gy/min, from 30 to 40 Gy/min, from 40 to 50 Gy/min, from 50 to 60 Gy/min, from 60 to 70 Gy/min, from 70 to 80 Gy/min, from 80 to 90 Gy/min, from 90 to 100 Gy/min, or greater than 100 Gy/min to the target (e.g., lesion).

In some embodiments, the shape of the RBS can provide a controlled projection of radiation (e.g., a therapeutic dose) onto the target, while allowing for the radiation dose to fall off quickly at the periphery of the target. This may help keep the radiation within a limited area/volume and may help prevent unwanted exposure of structures such as the optic nerve and/or the lens to radiation. Without wishing to limit the present invention to any theory or mechanism, it is believed that low areas/volumes of irradiation enables the use of higher dose rates, which in turn allows for faster surgery time and less complications.

The present invention further features a “fine positioning” surgical technique. For example, after inserting a cannula into a potential space under a Tenon's capsule of the eye of the patient, the surgeon observes the position of (through the patient's pupil via a “visual axis”) the treatment position of the cannula in a posterior pole of the eye, and adjust it accordingly to accurately localize it over the target. In some embodiments, the physician observes the position of the treatment position and adjusts it while the patient's eye is in a primary gaze position. A primary gaze position is when the patient looks straight ahead. In some embodiments, the physician observes the position of the treatment position and adjusts it while the patient's eye is in any one of the following position: elevated, depressed, adducted, elevated and adducted, elevated and abducted, depressed and adducted, and depressed and abducted. By seeing the position of the treatment position, the surgeon can adjust the cannula to position the treatment position over a target. In some embodiments, one of the advantages of the present “fine positioning” technique is that it allows for convenient and accurate placement of the RBS at the appropriate location behind the eye. In some embodiments, the fine positioning technique allows for placement of the cannula with precision.

Neovascular lesions of wet macula degeneration generally cannot be seen via indirect/direct ophthalmoscopy. In some embodiments, an angiogram (or other localizing technology such as optical coherence tomography, ultrasound) is performed, for example before the cannula is inserted between the Tenon's capsule and sclera. The angiogram may help locate the cannula and the target (e.g., lesion), and direct the cannula to the correct position over the target. For example, while localizing the target (e.g., lesion) via the surrounding landmarks and in reference to the previously obtained angiogram, the cannula may be directed to a precise position. In some embodiments, the localizing technology (e.g., angiogram) is a real-time procedure. In some embodiments, localizing technology is optical coherence tomography or ultrasound or other technology. In some embodiments, a photograph or video may be taken during the procedure to document the placement of the cannula.

Without wishing to limit the present invention to any theory or mechanism, it is believed that the system of the present invention is advantageous because it provides for improved positioning of the cannula around the eye, as well as the ability to pre-load the cannula easily. For example, the system geometry provides for a more robust addressable treatment area. The present invention has improved dosimetry. Improvements in dosimetry have traditionally driven better outcomes in many therapeutic applications in radiation treatment. The present invention has improved ergonomics and ease of use for the surgeon and also provides procedural staff with easier assembly or loading (e.g., of the RBS).

The present invention features a brachytherapy applicator system comprising: a cannula comprising a curved distal portion for placement around a portion of a globe of an eye, the distal portion has a radius of curvature from 9 to 15 mm and an arc length from 25 to 35 mm; and a curved proximal portion connected to the distal portion by an inflection point or a straight portion; and a radionuclide brachytherapy source (RBS) holder directly or indirectly connected to the distal portion, the RBS holder is adapted to hold a radionuclide brachytherapy source (RBS).

The present invention also features a system comprising: a cannula comprising: a curved distal portion for placement around a portion of a globe of an eye, the distal portion has a radius of curvature from 9 to 15 mm and an arc length from 25 to 35 mm; a curved proximal portion connected to the distal portion by an inflection point or a straight portion, the proximal portion has a radius of curvature from 1 mm to 500 mm. The system or cannula may feature the inflection point, which is where the distal portion and the proximal portions connect with each other. In some embodiments, the cannula further comprises a handle extending from the proximal portion. The handle lies on an axis such that the axis does not intersect with the distal portion. In some embodiments, the angle θ₁ between (i) a line l₃ tangent to the inflection point between the distal portion and the proximal portion and (ii) the proximal portion is between greater than about 0 degrees to about 180 degrees.

In some embodiments, at least a portion of the cannula is hollow. In some embodiments, at least a portion of the cannula is solid. In some embodiments, the distal portion and proximal portion are both solid.

In some embodiments, the distal portion has a radius of curvature of about 12 mm and the distal portion has an arc length of about 30 mm.

In some embodiments, the cannula further comprises a radionuclide brachytherapy source (RBS), e.g., an RBS disposed at a tip of the distal portion. In some embodiments, the tip of the distal portion has a diameter or width that is greater than that of the distal portion. In some embodiments, the handle comprises a radiation shielding pig for shielding a radionuclide brachytherapy source (RBS).

The cannula may further comprise a radionuclide brachytherapy source (RBS) holder directly or indirectly connected to the distal portion, the RBS holder comprises a cavity adapted to hold a radionuclide brachytherapy source (RBS), and a cap removably attachable to the RBS holder for sealing the cavity, wherein the cap comprises a head configuration to allow for engagement with a tool that can move the cap in a manner so as to secure the cap to the RBS holder and seal the cavity.

In some embodiments, the cannula has an outer cross sectional shape that is round or oval.

In some embodiments, at least a portion of the cannula is hollow, and the cannula has an internal cross sectional shape that is configured to allow a radionuclide brachytherapy source (RBS) to glide through. In some embodiments, at least a portion of the cannula is hollow, and the cannula has an internal cross sectional shape that is round or oval.

The present invention also features a brachytherapy applicator system comprising: a cannula comprising: a curved distal portion for placement around a portion of a globe of an eye, the distal portion has a radius of curvature from 9 to 15 mm and an arc length from 25 to 35 mm; and a curved proximal portion connected to the distal portion by an inflection point or a straight portion, the proximal portion has a radius of curvature from 1 mm to 500 mm; a radionuclide brachytherapy source (RBS) holder directly or indirectly connected to the distal portion, the RBS holder is adapted to hold a radionuclide brachytherapy source (RBS); and a handle directly connected to the proximal portion or indirectly connected to the proximal portion via a straight proximal portion, wherein the handle lies on an axis such that the axis does not intersect with the distal portion, the handle comprises a gripping component for helping a user hold the handle, an alignment component for providing a user a visual or tactile marking for determining orientation of the distal portion or RBS holder, or both a gripping component and alignment component.

The present invention also features a brachytherapy applicator system comprising: a cannula comprising: a curved distal portion for placement around a portion of a globe of an eye, the distal portion has a radius of curvature from 9 to 15 mm and an arc length from 25 to 35 mm; and a curved proximal portion connected to the distal portion by an inflection point or a straight portion, the proximal portion has a radius of curvature from 1 mm to 500 mm; and a radionuclide brachytherapy source (RBS) holder connected to the distal portion via a straight distal portion, the RBS holder is adapted to hold a radionuclide brachytherapy source (RBS).

The present invention also features a brachytherapy applicator system comprising: a cannula comprising: a curved distal portion for placement around a portion of a globe of an eye, the distal portion has a radius of curvature from 9 to 15 mm and an arc length from 25 to 35 mm; and a curved proximal portion connected to the distal portion by an inflection point or a straight portion, the proximal portion has a radius of curvature from 1 mm to 500 mm; and a radionuclide brachytherapy source (RBS) holder connected to the distal portion via a kink, the RBS holder is adapted to hold a radionuclide brachytherapy source (RBS).

The present invention also features a brachytherapy applicator system comprising: a cannula comprising: a curved distal portion for placement around a portion of a globe of an eye, the distal portion has a radius of curvature from 9 to 15 mm and an arc length from 25 to 35 mm; and a curved proximal portion connected to the distal portion by an inflection point or a straight portion, the proximal portion has a radius of curvature from 1 mm to 500 mm; and a radionuclide brachytherapy source (RBS) holder connected to the distal portion via a straight distal portion and kink, the kink being connected to the distal portion and the straight distal portion being connected to the RBS holder, the RBS holder is adapted to hold a radionuclide brachytherapy source (RBS).

The present invention also features a brachytherapy applicator system comprising: a cannula comprising: a curved distal portion for placement around a portion of a globe of an eye, the distal portion has a radius of curvature from 9 to 15 mm and an arc length from 25 to 35 mm; and a curved proximal portion connected to the distal portion by an inflection point or a straight portion, the proximal portion has a radius of curvature from 1 mm to 500 mm; a radionuclide brachytherapy source (RBS) holder directly or indirectly connected to the distal portion, the RBS holder is adapted to hold a radionuclide brachytherapy source (RBS); and a light system adapted to emit light from the RBS holder.

In some embodiments, an angle θ₁ between (i) a line l₃ tangent to the inflection point or straight portion and (ii) the proximal portion is from greater than 0 degrees to 180 degrees.

In some embodiments, the system comprises a handle directly connected to the proximal portion. In some embodiments, the system comprises a handle indirectly connected to the proximal portion via a straight proximal portion. In some embodiments, the handle lies on an axis such that the axis does not intersect with the distal portion. In some embodiments, the handle comprises a gripping component for helping a user hold the handle. In some embodiments, the gripping component comprises grooves, bumps, indentations, or scratches. In some embodiments, the handle further comprises an alignment component for providing a user a visual or tactile marking for determining orientation of the distal portion or RBS holder. In some embodiments, the cannula, the handle, or both the cannula and handle further comprise an alignment component for providing a user a visual or tactile marking for determining orientation of the distal portion or RBS holder). In some embodiments, the alignment component comprises a visual mark or visual distinction. In some embodiments, the alignment component comprises an indentation, a bump, or a ridge. In some embodiments, the alignment component is disposed on or in the distal portion of the handle.

In some embodiments, the RBS holder comprises a cavity adapted to hold an RBS. In some embodiments, the system comprises a cap for sealing the cavity. In some embodiments, the cap comprises a head configuration to allow for engagement with a tool that can move the cap in a manner so as to secure the cap to the RBS holder and seal the cavity. In some embodiments, the head configuration comprises at least one indentation or slot in a top surface of the cap. In some embodiments, the head configuration comprises a single slot, a pair of slots, a pair of indentations, a cruciform shaped screw drive or an internal hex. In some embodiments, the head configuration comprises at least one external side edge different from other external side edges. In some embodiments, the head configuration comprises an external hex. In some embodiments, the cap is a snap-on cap adapted to fit onto the RBS holder or within the RBS holder. In some embodiments, the cap is a pivot cap.

In some embodiments, the RBS holder is connected to the distal portion via a straight distal portion. In some embodiments, the RBS holder is connected to the distal portion via a kink. In some embodiments, the RBS holder is connected to the distal portion via a kink and a straight distal portion, wherein the kink is connected to the distal portion and the straight distal portion is connected to the RBS holder. In some embodiments, the straight distal portion engages a socket in the RBS holder. In some embodiments, the kink engages a socket in the RBS holder. In some embodiments, the straight distal portion has a length from 0.1 mm to 25 mm. In some embodiments, the kink has a radius of curvature from 1 to 500 mm. In some embodiments, the kink has an arc length from 0.1 to 20 mm.

In some embodiments, the system comprises a light system adapted to emit light from the RBS holder or distal portion. In some embodiments, the light system comprises a light emitting diode (LED) disposed in the RBS holder. In some embodiments, the light system comprises a fiber optic light wire disposed on at least a bottom surface of the RBS holder, wherein light is emitted from a tip of the fiber optic light wire. In some embodiments, the fiber optic light wire extends through the distal portion and proximal portion. In some embodiments, the fiber optic light wire extends through the distal portion and proximal portion and the handle. In some embodiments, the fiber optic light wire connects to a light source. In some embodiments, the light system is powered by a source connected via the proximal portion. In some embodiments, the light system is powered by a source located in the handle of the system. In some embodiments, the light system is powered by a source located external to the system.

In some embodiments, an RBS is loaded into the RBS holder prior to insertion of the system in a patient.

The present invention also features a cannula with a fixed shape, wherein the cannula comprises a distal portion for placement around a portion of a globe of an eye; a proximal portion connected to the distal portion via an inflection point; and a handle extending from the proximal portion, the handle lies on an axis such that the axis does not intersect with the distal portion. In some embodiments, the distal portion has a shape of an arc formed from a connection between two points located on an ellipsoid, the ellipsoid having an x-axis dimension “a”, a y-axis dimension “b,” and a z-axis dimension “c,” wherein “a” is between about 0 to 1 meter, “b” is between about 0 to 1 meter, and “c” is between about 0 to 1 meter. In some embodiments, the proximal portion has a shape of an arc formed from a connection between two points on an ellipsoid, the ellipsoid having an x-axis dimension “d”, a y-axis dimension “e,” and a z-axis dimension “f,” wherein “d” is between about 0 to 1 meter, “e” is between about 0 to 1 meter, and “f” is between about 0 to 1 meter. In some embodiments, the angle θ₁ between (i) a line l₃ tangent to the inflection point between the distal portion and the proximal portion and (ii) the proximal portion is between greater than about 0 degrees to about 180 degrees.

In some embodiments, “a” is between about 0 to 50 mm, “b” is between about 0 and 50 mm, and “c” is between about 0 and 50 mm. In some embodiments, “d” is between about 0 to 50 mm, “e” is between about 0 and 50 mm, and “f” is between about 0 and 50 mm. In some embodiments, the inflection point creates a soft bend between the distal portion and the proximal portion. In some embodiments, the distal portion has an arc length of between about 25 to 35 mm. In some embodiments, the proximal portion has an arc length of between about 10 to 75 mm.

The present invention also features methods and devices for applying radiation (e.g., beta radiation) for the treatment of diseases including but not limited to wet age-related macular degeneration. The cannula size is small, which allows for minimally-invasive surgery by making a small incision in the conjunctiva and inserting the cannula under Tenon's membrane along the sclera. No dissection or manipulation of the globe is necessary. In some cases, the tip of the device may be held against the sclera, which may help to control the deposition of the radiation dose (e.g., to within a fraction of a millimeter).

In some embodiments, the devices and methods of the present invention deliver radiation to the episcleral surface. In some embodiments, the radiation source is used within a disposable applicator. In some embodiments, the dose is 24 Gy and is given to the target (e.g., wet AMD lesion) over the course of 5 to 7 minutes, depending on the source strength, e.g., 555 to 740 MBq (15-20 mCi). The present invention also features methods of determining the dose delivered to the lesion and nearby normal tissues.

The present invention features a brachytherapy devices for delivery of radiation. In some embodiments, the device comprises a curved cannula divided into a distal portion and a proximal portion, wherein the distal portion is for placement around a portion of a globe of an eye. In some embodiments, the distal portion has a radius of curvature between about 9 to 15 mm and an arc length between about 25 to 35 mm. The device may further comprise a radionuclide brachytherapy source (RBS). In some embodiments, the device comprises a shield compartment for temporarily housing the RBS. In some embodiments, the shield compartment is connected to or part of the distal portion of the cannula. In some embodiments, the shield compartment is part of or connected to the proximal portion of the cannula. In some embodiments, the RBS can be advanced to a tip region of the distal portion of the cannula. In some embodiments, the RBS is fixed in a place in the cannula. For example, in some embodiments, the RBS is disposed in the tip of the cannula (e.g., the tip of the distal portion of the cannula.

In some embodiments, the RBS is encapsulated. In some embodiments, the RBS is encapsulated in stainless steel. In some embodiments, the RBS comprises four enamel beads, each bead is impregnated with Sr-90. In some embodiments, each bead is about 0.5 mm in diameter. In some embodiments, the RBS comprises Sr-90. In some embodiments, the RBS can deliver a dose of about 24 Gy to a target.

The present invention also features a method of irradiating a target of an eye in a patient, said method comprising inserting a cannula into a potential space under a Tenon's capsule of the eye of the patient, the cannula having a radionuclide brachytherapy source (RBS) at a treatment position, wherein the RBS is positioned over the target and the RBS irradiates the target.

The present invention also features a method of irradiating a target of an eye in a patient. The method comprises inserting a cannula into a potential space under the Tenon's capsule. The cannula comprises a radionuclide brachytherapy source (RBS) at a treatment position, whereby the RBS is positioned over the target as shown. The RBS irradiates the target. In some embodiments, the treatment position is a location on or within the cannula (e.g., the middle of the cannula, along the length or a portion of the length of the cannula, near the end of the cannula). In some embodiments, the treatment position comprises a window on the cannula. In some embodiments, the treatment position is configured to receive an RBS. In some embodiments, an indentation tip and/or a light source is disposed at the treatment position.

In some embodiments, the Tenon's capsule guides the insertion of the cannula and provides positioning support for the cannula. In some embodiments, the target is a lesion associated with the retina. In some embodiments, the target is located on the vitreous side of the eye. In some embodiments, the target (e.g., lesion) is a benign growth or a malignant growth.

In some embodiments, method comprises inserting a cannula between the Tenon's capsule and the sclera of the eye, for example at the limbus, a point posterior to the limbus of the eye, a point between the limbus and the fornix. In some embodiments, any appropriate cannula may be used in accordance with the present invention for the subtenon procedure. In some embodiments, cannulas that may be used in accordance with the present invention include flexible cannulas, fixed shape cannulas (or a combination of a flexible and fixed shape cannula), and cannulas which are tapered to provide a larger circumferential surface in the portion of the cannula which remains in the Tenon's capsule upon insertion, thereby providing additional positioning support to maintain the cannula over the target. In some embodiments, the arc length of the distal portion of the cannula is suitably of sufficient length to penetrate the Tenon's capsule and extend around the outside of the globe of the eye to a distal end position in close proximity to the macular target.

In some embodiments, the cannula employed in the inventive subtenon procedure comprises a distal portion, which is a portion of the cannula that is placed around a portion of the globe of the eye. The cannula has a radionuclide brachytherapy source (“RBS”) at a treatment position (e.g., in the middle of the cannula, near the end, in the middle, along the length of the cannula). The cannula may be “preloaded” with an RBS or “afterloaded”. For example, in some embodiments, the RBS is loaded into the cannula before the cannula is inserted. For example, in U.S. Pat. No. 7,070,554 to White, the brachytherapy device comprises a “preloaded” radiation source, i.e., a radiation source affixed at the tip of the device prior to the insertion of the device into the eye. In some embodiments, the RBS is loaded into the cannula after the cannula is inserted. The method further comprises positioning the RBS over the sclera portion that corresponds with the target (e.g., lesion), and the RBS irradiates the target (e.g., lesion) through the sclera.

The cannula may be of various shapes and sizes and constructed from a variety of materials. In some embodiments, the cannula is a fixed shape cannula. In some embodiments, the cannula is a flexible cannula, including an endoscope-like device. In some embodiments, the cannula is tapered (e.g., a larger circumferential area in the portion which remains in the Tenon's capsule upon insertion.

In some embodiments, the target is a lesion associated with the retina. In some embodiments, the target (e.g., lesion) is a neovascular lesion.

Neovascular lesions of wet macula degeneration generally cannot be seen via indirect/direct ophthalmoscopy. In some embodiments, an angiogram (or other localizing technology such as optical coherence tomography, ultrasound) is performed, for example before the cannula is inserted between the Tenon's capsule and sclera. The angiogram may help locate the cannula and the target (e.g., lesion), and direct the cannula to the correct position over the target. For example, while localizing the target (e.g., lesion) via the surrounding landmarks and in reference to the previously obtained angiogram, the cannula may be directed to a precise position. In some embodiments, the cannula comprises a window and/or an orifice, and the window/orifice of the cannula can be placed directly behind the target (e.g., lesion). In some embodiments, a photograph or video may be taken during the procedure to document the placement of the cannula.

In some embodiments, an angiogram, optical coherence tomography, ultrasound, or other localizing technology is performed, for example after the cannula is inserted between the Tenon's capsule and sclera. The localizing technology (e.g., angiogram) may help locate the cannula and the target (e.g., lesion), and direct the cannula to the correct position over the target. For example, while visualizing the target (e.g., lesion) via the localizing technology (e.g., angiogram), the cannula may be directed to a precise position. In some embodiments, the cannula comprises a window and/or an orifice, and the window/orifice of the cannula can be placed directly behind the target (e.g., lesion). In some embodiments, the localizing technology (e.g., angiogram) is a real-time procedure. In some embodiments, localizing technology is optical coherence tomography or ultrasound or other technology. In some embodiments, a photograph or video may be taken during the procedure to document the placement of the cannula.

The RBS can be constructed to provide any dose rate to the target. In some embodiments, the RBS provides a dose rate of between about 0.1 to 1 Gy/min, between about 1 to 10 Gy/min, between about 10 to 20 Gy/min, between about 20 to 30 Gy/min, between about 30 to 40 Gy/min, between about 40 to 50 Gy/min, between about 50 to 60 Gy/min, between about 60 to 70 Gy/min, between about 70 to 80 Gy/min, between about 80 to 90 Gy/min, between about 90 to 100 Gy/min, or greater than 100 Gy/min to the target (e.g., lesion).

The present invention also features a method of irradiating a target (e.g., lesion associated with the retina) of an eye in a patient. The method comprises inserting a cannula into the potential space under the Tenon's capsule (e.g., between the Tenon's capsule and the sclera) of the eye. In some embodiments, the cannula is inserted at the limbus, a point posterior to the limbus, or a point between the limbus and the fornix. In some embodiments, the cannula comprises a distal portion (e.g., a portion of the cannula that is placed over a portion of the globe of the eye). In some embodiments, the distal portion of the cannula is placed on or near the sclera behind the target (e.g., a lesion on the retina). A radionuclide brachytherapy source (RBS) is advanced through the cannula, for example to the treatment position (e.g., in the middle of the cannula, near a tip/end of distal portion), via a means for advancing the RBS. (In some embodiments, the means for advancing the RBS comprises a guide wire. In some embodiments, the means for advancing the RBS comprises a ribbon). The target is exposed to the RBS. The RBS may be loaded before the cannula is inserted or after the cannula is inserted.

The cannula may be constructed in various shapes and sizes. In some embodiments, the distal portion is designed for placement around a portion of the globe of the eye. In some embodiments, the distal portion has a radius of curvature between about 9 to 15 mm and an arc length between about 25 to 35 mm. In some embodiments, the cannula further comprises a proximal portion having a radius of curvature between about the inner cross-sectional radius of the cannula and about 1 meter. In some embodiments, the cannula further comprises an inflection point, which is where the distal portion and the proximal portions connect with each other. In some embodiments, the angle θ₁ between the line l₃ tangent to the globe of the eye at the inflection point and the proximal portion is between greater than about 0 degrees to about 180 degrees.

The present invention also features a hollow cannula with a fixed shape. The cannula comprises a distal portion for placement around a portion of the globe of an eye, wherein the distal portion has a radius of curvature between about 9 to 15 mm and an arc length between about 25 to 35 mm. The cannula further comprises a proximal portion having a radius of curvature between about the inner cross-sectional radius of the cannula and about 1 meter. The cannula further comprises an inflection point, which is where the distal portion and the proximal portions connect with each other. In some embodiments, the angle θ₁ between the line l₃ tangent to the globe of the eye at the inflection point and the proximal portion is between greater than about 0 degrees to about 180 degrees.

In some embodiments, once the distal end of the distal portion is positioned within the vicinity of the target, the proximal portion is curved away from the visual axis as to allow a user to have direct visual access in the eye.

The present invention also features a cannula with a fixed shape. The cannula comprises a distal portion for placement around a portion of a globe of an eye and a proximal portion connected to the distal portion via an inflection point. In some embodiments, the distal portion has a shape of an arc formed from a connection between two points located on an ellipsoid, wherein the ellipsoid has an x-axis dimension “a”, a y-axis dimension “b,” and a z-axis dimension “c.” In some embodiments, “a” is between about 0 to 1 meter, “b” is between about 0 to 1 meter, and “c” is between about 0 to 1 meter. In some embodiments, the proximal portion has a shape of an arc formed from a connection between two points on an ellipsoid, wherein the ellipsoid has an x-axis dimension “d”, a y-axis dimension “e,” and a z-axis dimension “f.” In some embodiments, “d” is between about 0 to 1 meter, “e” is between about 0 to 1 meter, and “f” is between about 0 to 1 meter. In some embodiments, the angle θ₁ between the line l₃ tangent to the globe of the eye at the inflection point and the proximal portion is between greater than about 0 degrees to about 180 degrees.

The present invention further features a “fine positioning” surgical technique. For example, after inserting a cannula into a potential space under a Tenon's capsule of the eye of the patient, the surgeon observes the position of (through the patient's pupil via a “visual axis”, see for example FIG. 7) the treatment position of the cannula in a posterior pole of the eye, and adjust it accordingly to accurately localize it over the target. In some embodiments, the physician observes the position of the treatment position and adjusts it while the patient's eye is in a primary gaze position. A primary gaze position is when the patient looks straight ahead. In some embodiments, the physician observes the position of the treatment position and adjusts it while the patient's eye is in any one of the following position: elevated, depressed, adducted, elevated and adducted, elevated and abducted, depressed and adducted, and depressed and abducted. By seeing the position of the treatment position, the surgeon can adjust the cannula to position the treatment position over a target. In some embodiments, one of the advantages of the present “fine positioning” technique is that it allows for convenient and accurate placement of the RBS at the appropriate location behind the eye. In some embodiments, the fine positioning technique allows for placement of the cannula with millimetric precision.

In some embodiments, the inventive methods of the present invention can be performed under general or local anesthesia (e.g., retro- or peribulbar) to the eye. When a general or local anesthesia is administered to the patient's eye, the eye will be in a primary position, and the patient has no motor movement of the eye. Accordingly, after the general or local anesthesia, the patient's eye will be in a primary gaze position, i.e., looking straight forward. The present inventive surgical methods and device allow for the surgeon to administer the radiation accurately to the target in the eye while the eye is in a primary gaze position. In some embodiments, the advantage of being able to perform the treatment while the patient's eye is in a primary gaze position is that it does not require the surgeon to perform additional surgical steps to secure the eye to a non-primary gaze position, as such securing steps may traumatize the eye.

The present invention also features a method of delivering radiation to an eye. The method comprises irradiating a target (e.g., a lesion associated with the retina, a target on the vitreous side of the eye, a benign growth, a malignant growth) from an outer surface of the sclera. In some embodiments, the target receives a dose rate of greater than about 10 Gy/min.

The present invention also features a method of irradiating a target (e.g., a target/lesion associated with the retina) of an eye in a patient. The method comprises placing a radionuclide brachytherapy source (RBS) at or near a portion of the eye (e.g., sclera) that corresponds with the target. The RBS irradiates the target through the sclera, wherein more than 1% of the radiation from the RBS is deposited on a tissue at or beyond a distance of 1 cm from the RBS. In some embodiments, about 1% to 15% of the radiation from the RBS is deposited on a tissue or beyond a distance of 1 cm from the RBS. In some embodiments, about less than 99% of the radiation from the RBS is deposited on a tissue at a distance less than 1 cm from the RBS.

The methods of the present invention also allow for delivering a smaller volume/area of radiation as compared to other procedures. For example, a radionuclide brachytherapy source (“RBS”) in the shape of a disk can provide a controlled projection of radiation (e.g., a therapeutic dose) onto the target, while allowing for the radiation dose to fall off quickly at the periphery of the target. This keeps the radiation within a limited area/volume and may help prevent unwanted exposure of structures such as the optic nerve and/or the lens to radiation. Without wishing to limit the present invention to any theory or mechanism, it is believed that low areas/volumes of irradiation enables the use of higher dose rates, which in turn allows for faster surgery time and less complications.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

FIG. 1A shows a perspective view of a brachytherapy system (100) of the present invention comprising a cannula (105) with a distal portion (110) and proximal portion (120) separated by a straight portion (132). A straight proximal portion (134) extends from the end of the proximal portion (120) and connects to the handle (140). A light source (152) (a fiber optic wire) extends through the handle (140) and cannula (105) to the RBS holder (230) at the tip of the distal portion (110). The RBS holder (230) is adapted to hold an RBS. The RBS holder (230) is attached to a straight distal portion (136) disposed at the tip of the distal portion (110).

FIG. 1B shows a detailed view of the RBS holder (230) with a cap (248) (e.g., a removable cap). A well or cavity (232) is disposed in the RBS holder (230) for accepting the RBS. In some embodiments, a gasket is sandwiched between the RBS holder (230) and cap (248).

FIG. 2A shows a detailed view of the cannula (105) of the system (100) of the present invention. Note in some embodiments, the RBS holder (230) is fixedly attached to the distal portion (110). In some embodiments, the RBS holder (230) is attachable (e.g., removably attached) to the distal portion (110).

FIG. 2B shows an alternative system (100) of the present invention wherein the cannula (105) comprises a distal portion (110) and a proximal portion (120) separated by an inflection point (130). Note that the handle (140) lies on an axis that does not intersect with the tip of the distal portion (110) of the cannula. Note curve “a” refers to the curve of the distal portion (110), e.g., the arc length and radius of curvature, and curve “b” refers to the curve of the proximal portion (120), e.g., the arc length and radius of curvature.

FIG. 3 shows a side view of the cannula (105) of FIG. 2A without the RBS holder (230). Note that the distal portion (110) (and RBS holder) does not cross or intersect with the axis of the handle or straight proximal portion (134). Without wishing to limit the present invention to any theory or mechanism, this configuration may be helpful for providing better visualization for a physician (e.g., the handle does not obstruct the visual axis for the physician).

FIG. 4 is an in-use view of the system (100) of FIG. 1A.

FIG. 5A is a bottom view of the RBS holder (230) of the present invention. Note the light emitting component (151) (e.g., the tip of the fiber optic wire (152)) exposed in the slot (153) at the bottom surface (230b) of the RBS holder (230).

FIG. 5B shows a cross-sectional view of a system (100) of the present invention. A fiber optic wire (152) (light emitting component (151)) extends through the cannula (105) and into or on the bottom surface (230b) of the RBS holder (230), e.g., in a slot (153) in the bottom surface (230b) of the RBS holder (230). The present invention is not limited to this configuration. In this example, a fiber optic wire carries the light to the tip. The tip functions as the light emitting component (151). The light emitting component (151) may alternatively be an LED or other appropriate light emitting component. In some embodiments, component (152) my refer to a fiber optic wire or a power lead to the LED (if the LED was the light emitting component)>In some embodiments, the power lead to the LED may extend through the slot (153). Also shown is the straight distal portion (136) engaged with the RBS holder (230), e.g., inserted into a socket (231) in the RBS holder.

FIG. 5C shows an in-use view of the system of the present invention, with a light system (150), wherein light is emitted from the light emitting component (151) (e.g., the tip of the fiber optic wire (152).

FIG. 6A, FIG. 6B, FIG. 6C, FIG. 6D, FIG. 6E, FIG. 6F, FIG. 6G, FIG. 6H, FIG. 6I, FIG. 6J, FIG. 6K, and FIG. 6L show alternative RBS holders and caps. The present invention is not limited to those shown these figures, nor the other configurations shown or described herein.

FIG. 7 shows the insertion of the cannula into the eye. The distal portion (110) and proximal portion (120) are configured such that the handle 140 and/or straight proximal portion (132) are out of the visual axis (dotted line) of the physician and the patient. Tenon's capsule a layer of tissue running from the limbus anteriorly to the optic nerve posteriorly. The Tenon's capsule is surrounded anteriorly by the bulbar conjunctiva that originates at the limbus and reflects posteriorly into the tarsal conjunctiva at the conjunctival fornix. Note the configuration of the tip of the cannula is different from that of FIG. 1A.

FIG. 8 shows an example of angle θ₁ 425 which is between line l₃ 420. Line l₃ (420) is tangent to the inflection point (130) and/or the straight proximal portion (132) (or also tangent to the globe of the eye at the inflection point (130) and/or straight proximal portion (132)).

FIG. 9 shows an example of an ellipsoid with the x-axis, y-axis, and z-axis. The distal portion may have the shape of an arc formed from a connection between two points on such an ellipsoid, wherein the x-axis dimension is “a”, the y-axis dimension is “b,” and the z-axis dimension is “c.” The proximal portion may have the shape of an arc formed from a connection between two points on such an ellipsoid, wherein the x-axis dimension is “d”, the y-axis dimension is “e,” and the z-axis dimension is “f.”

FIG. 10 is a detailed view of an example of a radionuclide brachytherapy source (RBS) (or radiation source) at the tip of the cannula of the device of the present invention.

FIG. 11 is a diagram showing the relative dose distribution in Gy/min at 1.5 mm distance from the source. The isodose lines are normalized to the central point.

FIG. 12 is a diagram showing the relative dose distribution in Gy/min at 3 mm distance from the source. The isodose lines are normalized to the central point at 1.5 mm depth.

FIG. 13 is a diagram showing the several central axis dose determinations on different days and the two different techniques for setting the depths. The dose rate is shown as a function of depth from the source center for the several measurements conducted, the one determination at 2.0 by the manufacturer, and the MCNPX calculations normalized to 8.9 Gy/min at 2.0 mm. Experimental dose rates were determined from the dose measured from an exposure divided by the time of the exposure. The label “T” refers to measurement in the treatment configuration (cylindrical phantom) while “L” refers to the lateral measurement using the side of the cannula on the flat phantom.

FIG. 14 is a diagram showing isodose lines at 2.7 mm determined experimentally and analyzed by hand. The dose rates are determined from the absolute doses measured for this exposure divided by the time of the exposure.

### DESCRIPTION OF PREFERRED EMBODIMENTS

Following is a list of elements corresponding to a particular element referred to herein:

100 brachytherapy applicator system

101 sclera

102 Tenon's capsule

105 cannula

110 distal portion of cannula

120 proximal portion of cannula

130 inflection point of cannula

132 straight portion between distal and proximal portions

134 straight proximal portion between proximal portion and handle

136 straight distal portion between distal portion and RBS holder

138 kink

140 handle

143 alignment component

144 gripping component

150 light system

151 light emitting component (e.g. tip of fiber optic wire, LED, etc.)

152 fiber optic light wire (or could be a power lead, e.g., to an LED, etc.)

153 slot in bottom surface of RBS holder

180 radionuclide brachytherapy source (RBS)

230 RBS holder (disc applicator)

230b bottom surface of RBS holder

231 socket

232 well/cavity for RBS

248 cap

249 head configuration

250 shield

420 line l₃

421 line l₄

425 angle θ₁

As used herein, the term “about” means plus or minus 10% of the referenced number. For example, an embodiment wherein an angle is about 50 degrees includes an angle between 45 and 55 degrees.

### The Eye

The mammalian eye is a generally spherical structure that performs its visual function by forming an image of an exterior illuminated object on a photosensitive tissue, the retina. The basic supporting structure for the functional elements of the eye is the generally spherical tough, white outer shell, the sclera, which is comprised principally of collagenous connective tissue and is kept in its spherical shape by the internal pressure of the eye. Externally the sclera is surrounded by the Tenon's capsule (fascia bulbi), a thin layer of tissue running from the limbus anteriorly to the optic nerve posteriorly. The Tenon's capsule is surrounded anteriorly by the bulbar conjunctiva, a thin, loose, vascularized lymphatic tissue that originates at the limbus and reflects posteriorly into the tarsal conjunctiva at the conjunctival fornix. Anteriorly the sclera joins the cornea, a transparent, more convex structure. The point where the sclera and cornea is called the limbus. The anterior portion of the sclera supports and contains the elements that perform the function of focusing the incoming light, e.g., the cornea and crystalline lens, and the function of regulating the intensity of the light entering the eye, e.g., the iris. The posterior portion of the globe supports the retina and associated tissues.

In the posterior portion of the globe (referred to herein as the “posterior portion of the eye”) immediately adjacent the interior surface of the sclera lays the choroid, a thin layer of pigmented tissue liberally supplied with blood vessels. The portion of the choroid adjacent its interior surface is comprised of a network of capillaries, the choriocapillaris, which is of importance in the supply of oxygen and nutrients to the adjacent layers of the retina. Immediately anterior to the choroid lies the retina, which is the innermost layer of the posterior segment of the eye and receives the image formed by the refractive elements in the anterior portion of the globe. The photoreceptive rod and cone cells of the retina are stimulated by light falling on them and pass their sensations via the retinal ganglion cells to the brain. The central region of the retina is called the macula. It is roughly delimited by the superior and inferior temporal branches of the central retina artery, and it is mostly responsible for color vision, contrast sensitivity and shape recognition. The very central portion of the macula is called the fovea and is responsible for fine visual acuity.

### Sub-Tenon Delivery of a Radionuclide Brachytherapy Source (RBS) to Posterior of Eye Globe

The present invention features brachytherapy systems and methods, e.g., methods (and systems) for minimally-invasive delivery of radiation to the posterior portion of the eye. Without wishing to limit the present invention to any theory or mechanism, it is believed that the sub-tenon method of delivering radiation to the posterior portion of the eye of the present invention is advantageous for several reasons. For example, the sub-tenon procedure is minimally invasive and does not require extensive surgical dissections. Thus, this unique procedure is faster, easier, and will present fewer side effects and/or complications the prior art methods that otherwise require dissections. Moreover, the sub-tenon method may allow for simple office-based procedures with faster recovery times.

The sub-tenon method also allows for the tenon's capsule and other structures (e.g., sclera) to help guide and hold the device in place when in use. Keeping the cannula in a fixed location and at a distance from the target during the treatment reduces the likelihood of errors and increases the predictability of dose delivery. In an intravitreal approach (e.g., irradiating the target area by directing the radiation from within the vitreous chamber from anteriorly to the retina of the eye back towards the target), a physician is required to hold the device in a fixed location and a fixed distance from the target in the spacious vitreous chamber. It may be difficult for the physician to hold precisely that position for any length of time. Furthermore, it is generally not possible for the physician/surgeon to know the exact distance between the probe and the retina; he/she can only estimate the distance.

The methods of the present invention direct radiation from the posterior side of the eye forwardly to a target; radiation is shielded in the back. Without wishing to limit the present invention to any theory or mechanism, it is believed that these methods will spare the patient from receiving ionizing radiation in the tissues behind the eye and deeper than the eye. A pre-retinal approach (e.g., irradiating the target area by directing the radiation from the anterior side of the retina back toward the target) irradiates the anterior structures of the eye (e.g., cornea, iris, ciliary body, lens) and has the potential to irradiate the tissues deeper than the lesion, such as the periorbital fat, bone, and the brain. An intravitreal radiation approach also has the potential to irradiate the tissues deeper than the lesion (e.g., periorbital fat, bone, brain) and also, in a forward direction, the lens, ciliary body and cornea.

Prior to the present invention, radiotherapy as applied to the eye generally involves invasive eye surgeries. For example, an authoritative report in the radiation therapy industry known as the “COMS study” discloses a protocol that employs an invasive surgical procedure to dissect the periocular tissues and place the brachytherapy device. This is unlike the presently inventive minimally invasive subtenon method.

The prior art has disclosed a number of brachytherapy devices and methods of using same for irradiating a lesion from behind the eye. However, these techniques do not employ the minimally invasive subtenon approach of the present invention. Upon reading the disclosures of the prior art, one of ordinary skill would easily recognize that the procedure being disclosed is quadrant dissection approach or a retro-bulbar intra-orbital approach, neither of which is the minimally invasive subtenon approach.

The present invention features a method of introducing radiation to the posterior portion of the eye in a minimally-invasive manner (by respecting the intraocular space). Generally, the method comprises irradiating from the outer surface of the sclera (e.g., under Tenon's capsule) to irradiate a target. The target may be the macula, the retina, the sclera, and/or the choroid. In some embodiments, the target may be on the vitreous side of the eye. In some embodiments, the target is a neovascular lesion. In some embodiments, the target receives a dose rate of radiation of greater than about 10 Gy/min.

The methods of the present invention may feature introducing a portion of a cannula of a brachytherapy system (100) (the cannula comprising an RBS) to the posterior portion of the eye between the Tenon's capsule and the sclera and exposing the posterior portion of the eye to the radiation. In some embodiments, the method further comprises the step of exposing the target (e.g., macula) of the eye to the radiation. In some embodiments, the method comprises targeting a neovascular growth in the macula.

In some embodiments, the RBS is placed in the subtenon space in close proximity to the portion of the sclera that overlays a portion of choroid and/or retina affected by an eye condition (e.g., WAMD, tumor). As used herein, a RBS that is placed “in close proximity” means that the RBS is about 0 mm to about 10 mm from the surface of the sclera. In some embodiments, the radiation irradiates through the sclera to the choroid and/or retina.

In some embodiments, the step of inserting the cannula (of the system (100)) between the Tenon's capsule and the sclera further comprises inserting the cannula into the superior temporal quadrant of the eye. In some embodiments, the step of inserting the cannula between the Tenon's capsule and the sclera further comprises inserting the cannula into the inferior temporal quadrant of the eye. In some embodiments, the step of inserting the cannula between the Tenon's capsule and the sclera further comprises inserting the cannula into the superior nasal quadrant of the eye. In some embodiments, the step of inserting the cannula between the Tenon's capsule and the sclera further comprises inserting the cannula into the inferior nasal quadrant of the eye.

In some embodiments, the distance from the RBS to the target is from 0.4 to 2.0 mm. In some embodiments, the distance from the RBS to the target is from 0.4 to 1.0 mm. In some embodiments, the distance from the RBS to the target is from 1.0 to 1.6 mm. In some embodiments, the distance from the RBS to the target is from 1.6 to 2.0 mm. In some embodiments, the distance from the RBS to the target is between 0.0 to 10.0 mm. In some embodiments, the distance from the RBS to the target is between 0.1 to 3.0 mm. In some embodiments, the distance from the RBS to the target is from 0.1 to 5.0 mm. In some embodiments, the distance from the RBS to the target is between 0.5 to 10 mm. In some embodiments, the distance from the RBS to the target is between 1 to 10 mm. In some embodiments, the distance from the RBS to the target is between 5 to 10 mm. The present invention is not limited to these ranges. For example, the distance from the RBS target may be more than 10 mm, e.g., from 10 to 20 mm, from 10 to 30 mm, etc., e.g., the distance may depend on the treatment and/or target.

The present methods may be effective for treating and/or managing a condition (e.g., an eye condition). For example, the present methods may be used to treat and/or manage wet (neovascular) age-related macula degeneration. The present methods are not limited to treating and/or managing wet (neovascular) age-related macular degeneration. For example, the present methods may also be used to treat and/or manage conditions including macula degeneration, abnormal cell proliferation, choroidal neovascularization, retinopathy (e.g., diabetic retinopathy, vitreoretinopathy), macular edema, and tumors (e.g., intra ocular melanoma, retinoblastoma).

Without wishing to limit the present invention to any theory or mechanism, it is believed that the novel subtenon methods of the present invention are advantageous over the prior art because they are less invasive (e.g., they do not invade the intraocular space), they require only local anesthesia, and they provide a quicker patient recovery time. For example, the technique of introducing radiation to the posterior portion of the eye by suturing a radioactive plaque on the sclera at the posterior portion of the eye requires a 360° peritomy (e.g., dissection of the conjunctiva), isolation of the four recti muscles and extensive manipulation of the globe. Furthermore, when the plaque is left in place and then removed a few days later, a second surgery is required. The methods of the present invention are easier to perform. Also, the intraocular method of exposing the posterior pole of the eye to radiation involves performing a vitrectomy as well as positioning and holding the radioactive probe in the preretinal vitreous cavity for a significant length of time without a stabilizing mechanism. This technique is difficult to perform, requires a violation of the intraocular space, and is prone to a number of possible complications such as the risk of retinal detachment, cataracts, glaucoma, and/or endophthalmitis. Because of the complexity of this technique, a fellowship in vitreoretina surgery is required. The methods of the present invention are easier to perform, minimally-invasive, and do not impose a risk of damage to the intraocular structures. Moreover, the methods of the present invention do not require additional vitreoretina fellowship training as these methods can be employed by any surgical ophthalmologist.

As used herein, the term “minimally-invasive” method means a method that does not require that an instrument be introduced into the intraocular space (anterior, posterior, or vitreous chamber) of the eye for delivery of a radioactive source to the posterior portion of the eye or a method that does not require the suturing of a radioactive plaque on the sclera or extensive conjunctiva peritomy. For example, the minimally-invasive methods of the present invention only require a small incision of conjunctiva and Tenon's capsule for inserting of a cannula comprising a RBS to the posterior portion of the eye. The preferred approach is through the superotemporal quadrant, however entrance through the supero nasal, the inferotemporal or the inferonasal quandrant can be employed.

In some embodiments, the area of sclera exposed to the radiation is about 0.1 mm to about 0.5 mm in diameter. In some embodiments, the area of sclera exposed to the radiation is about 0.5 mm to about 2 mm in diameter. In some embodiments, the area of sclera exposed to the radiation is about 2 mm to 3 mm in diameter. In some embodiments, the area of sclera exposed to the radiation is about 3 mm to 5 mm in diameter. In some embodiments, the area of sclera exposed to the radiation is about 5 mm to 10 mm in diameter. In some embodiments, the area of sclera exposed to the radiation is about 10 mm to 25 mm in diameter.

### Cannula of the Brachytherapy System

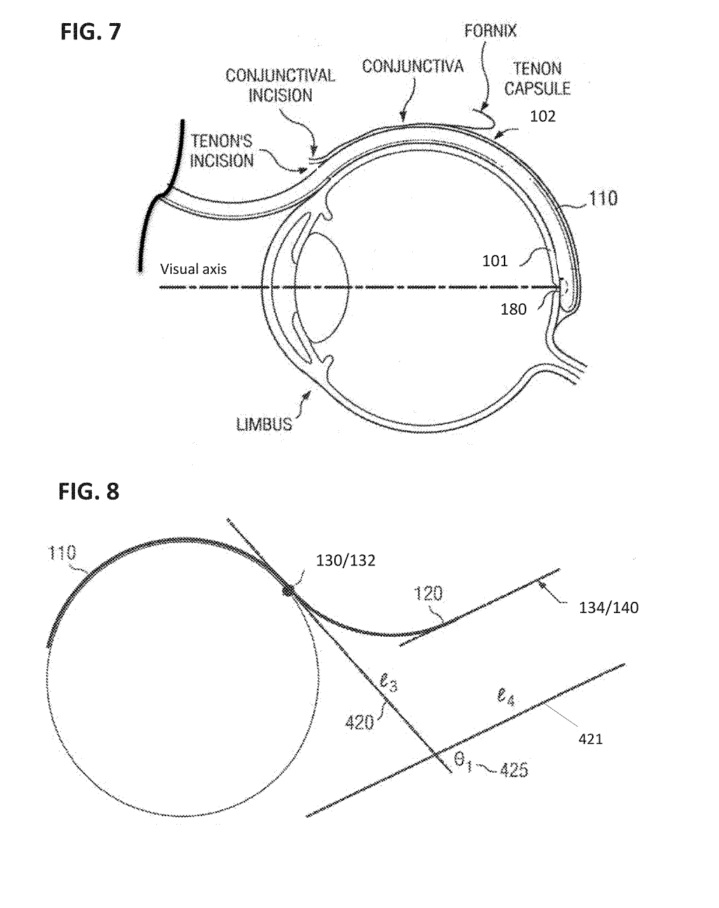

The present invention features a brachytherapy system (100) (e.g., comprising a cannula) for delivering a radionuclide brachytherapy source (RBS) to the back of the eye. In some embodiments, the system (100) comprises a cannula (105), wherein the cannula (105) comprises a distal portion (110) connected to a proximal portion (120). In some embodiments, the distal portion (110) and proximal portion (120) are connected via a straight portion (132). In some embodiments, the distal portion (110) and proximal portion (120) are connected via an inflection point (130). The distal portion (110) is for placement around a portion of the globe of the eye. In some embodiments, the distal portion (110) is inserted below the Tenon's capsule and above the sclera (see FIG. 7).

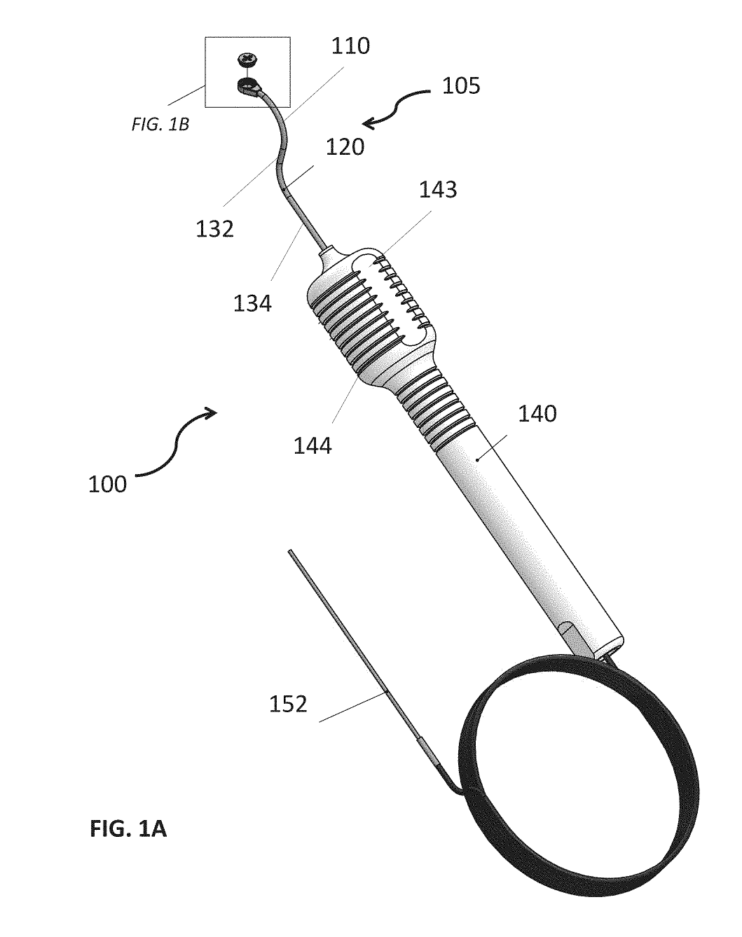

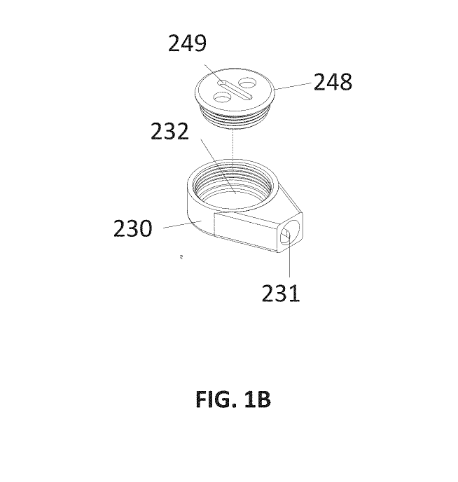

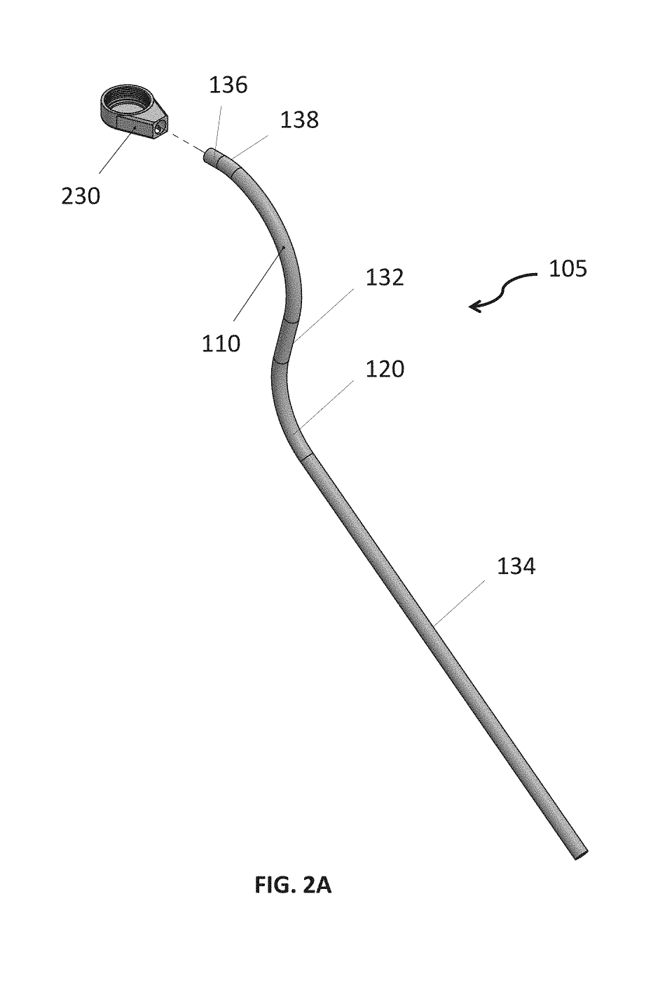

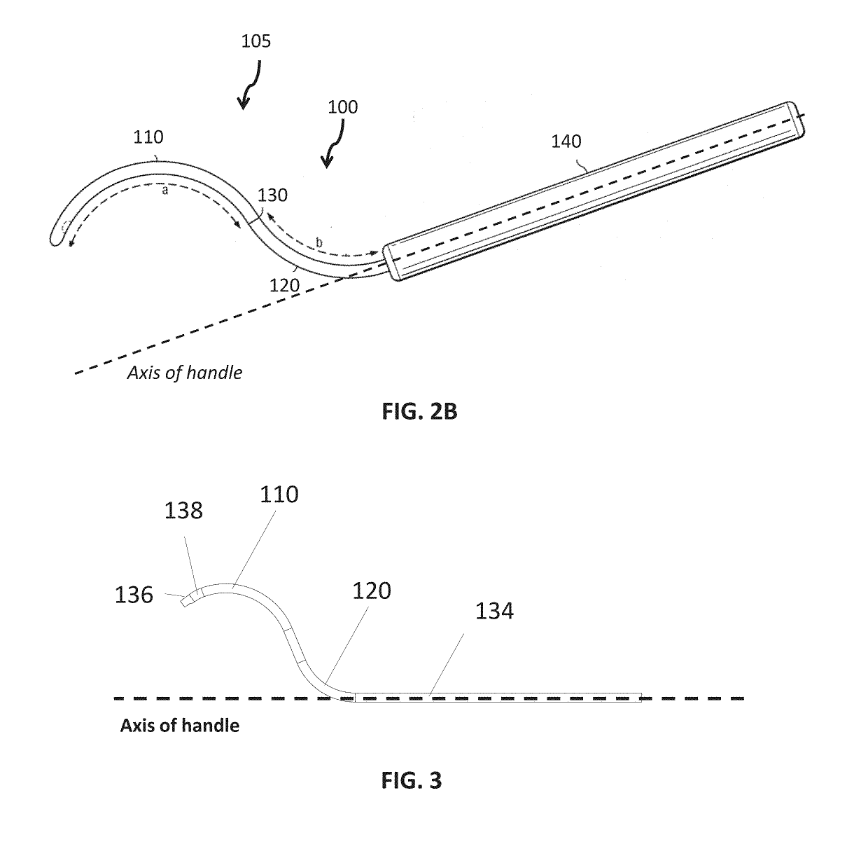

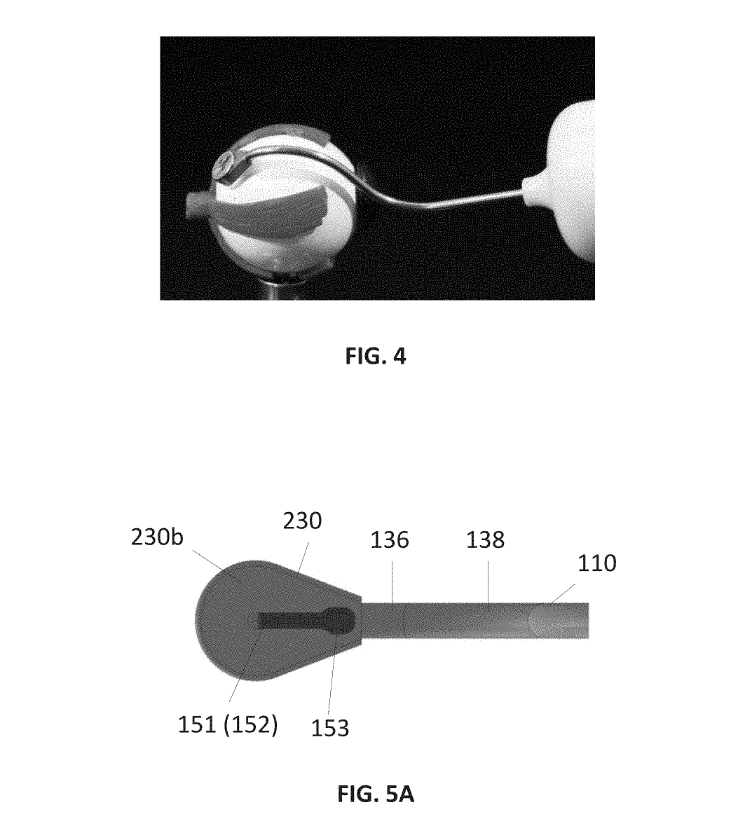

In some embodiments, the inflection point (130) and/or the straight portion (132) helps to position the proximal portion (120) of the cannula (105) away from the visual axis (see dotted line of FIG. 7) of the subject (e.g., patient) and of the user (e.g., physician) who inserts the system (100) (e.g., cannula (105), e.g., distal portion (110) of the cannula (105)) into a subject. FIG. 1A and FIG. 2A show examples of a cannula (105) wherein the proximal portion (120) and distal portion 9110) are connected via a straight portion (132). FIG. 2B shows an example of a cannula (105) wherein the proximal portion (120) and distal portion (110) are connected via an inflection point (130).

In some embodiments, the distal portion (110) has a radius of curvature from about 9 to 15 mm (e.g., 9 mm, 10 mm, 11 mm, 12 mm, 13 mm, 14 mm, 15 mm, or a dimension from 9 to 15 mm). In some embodiments, the distal portion (110) has an arc length from about 25 to 35 mm (e.g., 25 mm, 26 mm, 27 mm, 28 mm, 29 mm, 30 mm, 31 mm, 32 mm, 33 mm, 34 mm, 35 mm, or a dimension from 25 mm to 35 mm).

In some embodiments, the distal portion (110) has an arc length from 10 to 15 mm. In some embodiments, the distal portion (110) has an arc length from 15 mm to about 20 mm. In some embodiments, the distal portion (110) has an arc length from 20 mm to about 25 mm. In some embodiments, the distal portion (110) has an arc length from 25 mm to about 30 mm. In some embodiments, the distal portion (110) has an arc length from 30 mm to about 35 mm. In some embodiments, the distal portion (110) has an arc length from 35 mm to about 50 mm. In some embodiments, the distal portion (110) has an arc length from 50 mm to about 75 mm. In some embodiments, the arc length of the distal portion (110) may also serve to limit the depth of insertion of the system (100) (e.g., cannula) along the sclera, preventing the tip of the distal portion (110) from accidentally damaging posterior ciliary arteries or the optic nerve.

In some embodiments, the proximal portion (120) has a radius of curvature from 0.1 mm to 1 meter. In some embodiments, the proximal portion (120) has a radius of curvature from 1 mm to 500 mm. In some embodiments, the proximal portion (120) has a radius of curvature from 1 mm to 250 mm. In some embodiments, the proximal portion (120) has a radius of curvature from 1 mm to 100 mm. In some embodiments, the proximal portion (120) has a radius of curvature from 1 mm to 75 mm. In some embodiments, the proximal portion (120) has a radius of curvature from 1 mm to 50 mm. In some embodiments, the proximal portion (120) has a radius of curvature from 1 mm to 40 mm. In some embodiments, the proximal portion (120) has a radius of curvature from 1 mm to 30 mm. In some embodiments, the proximal portion (120) has a radius of curvature from 1 mm to 20 mm. In some embodiments, the proximal portion (120) has a radius of curvature from 1 mm to 10 mm. In some embodiments, the radius of curvature of the proximal portion (120) is constant. In some embodiments, the radius of curvature of the proximal portion (120) is variable.

In some embodiments, the proximal portion (120) has an arc length from 10 to 70 mm (e.g., 10 mm, 20 mm, 30 mm, 40 mm, 50 mm, 60 mm, 75 mm, or a dimension from 10 to 75 mm). In some embodiments, the proximal portion (120) has an arc length that is 10 mm or less, e.g., 10 mm, 9.5 mm, 9 mm, etc.

In some embodiments, a straight proximal portion (134) (and/or handle (140) extends from the proximal portion (120) of the cannula (105). In some embodiments, a straight distal portion (136) extends from the end of the distal portion (110) of the cannula (105).

As used herein, the term “arc length” of the distal portion (110) refers to the arc length measured from the tip of the distal portion (110) to the inflection point (130) or the straight portion (132) between the distal portion (110) and proximal portion (120). The term “arc length” of the proximal portion (120) refers to the arc length measured from the inflection point (130) or straight portion (132) and the end of the proximal portion (120), e.g., wherein the straight proximal portion (134) or handle (140) connects to the proximal portion (120). The term “radius of curvature” of the distal portion (110) or proximal portion (120) refers to the length of the radius of a circle/oval defined by the curve of the distal portion (110) or proximal portion (120), respectively.

In some embodiments, the radius of curvature of the distal portion (110) is constant. For example, the radius of curvature in the distal portion (110) may be a constant 12 mm. In some embodiments, the radius of curvature of the distal portion (110) is variable. For example, the radius of curvature in the distal portion (110) may be larger at the distal region and smaller at the middle region.

The distal portion (110) and the proximal portion (120) of the system (e.g., cannula) each have an outer diameter (as viewed from a vertical cross section). In some embodiments, the outer diameter of the distal portion (110) and/or proximal portion (120) is constant. In some embodiments, the outer diameter of the distal portion (110) and/or proximal portion (120) is variable. In some embodiments, the system (e.g., cannula, e.g., distal portion (110), proximal portion (120), straight portion (132), portions thereof, combinations thereof, etc.) has an outer cross sectional shape that is generally circular or round. In some embodiments, the system (e.g., cannula, e.g., distal portion (110), proximal portion (120), straight portion (132), portions thereof, combinations thereof, etc.) has an outer cross sectional shape that is oval, rectangular, egg-shaped, or trapezoidal.

In some embodiments, the average outer diameter of the vertical cross section of the distal portion (110) is from 0.1 mm and 0.4 mm. In some embodiments, the average outer diameter of the distal portion (110) is from 0.4 mm and 1.0 mm. In some embodiments, the average outer diameter of the distal portion (110) is about 0.9 mm. In some embodiments, the average outer diameter of the distal portion (110) is from 1.0 mm and 2.0 mm. In some embodiments, the average outer diameter of the distal portion (110) is from 2.0 mm and 5.0 mm. In some embodiments, the average outer diameter of the distal portion (110) is from 5.0 mm and 10.0 mm. In some embodiments, the average outer diameter of the vertical cross section of the distal portion (110) is about 0.4 mm. In some embodiments, the average outer diameter of the vertical cross section of the distal portion (110) is about 0.7 mm. In some embodiments, the average outer diameter of the distal portion (110) is about 0.9 mm. In some embodiments, the average outer diameter of the distal portion (110) is about 1.3 mm. In some embodiments, the average outer diameter of the distal portion (110) is about 1.7 mm. In some embodiments, the average outer diameter of the distal portion (110) is about 1.8 mm. In some embodiments, the average outer diameter of the distal portion (110) is about 2.1 mm. Note in some embodiments, the distal portion (110) is slightly rectangular, e.g., the distal portion (110) may not have a diameter necessarily but instead a width. In some embodiments, the width of the distal portion (110) may be from 0.5 mm to 1 mm. In some embodiments, the width of the distal portion (110) may be from 1 mm to 3 mm (e.g., 2 mm). In some embodiments, the width of the distal portion (110) may be from 1 mm to 5 mm (e.g., 4 mm). In some embodiments, the width of the distal portion (110) may be from 0.1 mm to 3 mm (e.g., 1 mm). In some embodiments, the width of the distal portion (110) may be from 0.2 mm to 2.5 mm (e.g., 1.8 mm).

In some embodiments, at least a portion of the cannula (105) is hollow. In some embodiments, at least a portion of the cannula (105) is solid. For example, in some embodiments, a portion of the cannula (105) is hollow so as to allow a fiber optic wire to extend through the cannula (105). The present invention is not limited to this configuration. For example, in some embodiments, the cannula (105) is completely solid.

As shown in FIG. 8, line l₃ (420) represents the line tangent to the inflection point (130) and/or limbus and/or the straight portion (132) between the proximal (120) and distal portion (110), etc.). Line l₃ (420) and line l₄ (421) (the straight proximal portion (134) of the system (100) or a line parallel to the straight proximal portion (134) of the system (100)) form angle θ₁ (425). The system (100) (e.g., cannula (105)) may be constructed in many ways; therefore angle θ₁ (425) may have various values. In some embodiments, the angle θ₁ (425) is from greater than about 0 (e.g., 0.1, 1, 5, 10, 15, 20, etc.) to 180 degrees. In some embodiments, if the cannula (105) bends through a larger angle, the value of angle θ₁ (425) is greater. In some embodiments, the angle θ₁ (425) between (i) the line l₃ (420) tangent to the inflection point (130) and/or the straight portion (132) of the cannula (105) and (ii) the straight proximal portion (134) and/or handle (140) is from greater than about 0 degrees (e.g., 0.1, 1, 5, 10, 15, 20, etc.) to about 180 degrees, e.g., from 0.1 degrees to 180 degrees, from 1 to 180 degrees, from 5 to 180 degrees, etc.

In some embodiments, angle θ₁ (425) is from 1 to 10 degrees. In some embodiments, angle θ₁ (425) is from 10 to 20 degrees. In some embodiments, angle θ₁ (425) is from 20 to 30 degrees. In some embodiments, angle θ₁ (425) is from 30 to 40 degrees. In some embodiments, angle θ₁ (425) is from 40 to 50 degrees. In some embodiments, angle θ₁ (425) is from 50 to 60 degrees. In some embodiments, angle θ₁ (425) is from 60 to 70 degrees. In some embodiments, angle θ₁ (425) is from 70 to 80 degrees. In some embodiments, angle θ₁ (425) is from 80 to 90 degrees. In some embodiments, angle θ₁ (425) is from 90 to 100 degrees. In some embodiments, angle θ₁ (425) is from 100 to (110) degrees. In some embodiments, angle θ₁ (425) is from (110) to 120 degrees. In some embodiments, angle θ₁ (425) is from 120 to 130 degrees. In some embodiments, angle θ₁ (425) is from 140 to 150 degrees. In some embodiments, angle θ₁ (425) is from 150 to 160 degrees. In some embodiments, angle θ₁ (425) is from 160 to 170 degrees. In some embodiments, angle θ₁ (425) is from 170 to 180 degrees.

In some embodiments, the distal portion (110) and the proximal portion (120) lie in the same plane. In some embodiments, the proximal portion (120) is off at an angle from the distal portion (110), for example the proximal portion (120) is rotated or twisted with respect to the distal portion (110) such that the distal portion (110) and the proximal portion (120) lie in different planes. In some embodiments, the distal portion (110) and proximal portion (120) can be rotated/twisted with respect to each other from −90° and +90°.

As previously discussed, in some embodiments, a straight portion (132) is disposed between the distal portion (110) and proximal portion (120). For example, the inflection point (130) may extend into a segment (straight portion (132)) between the distal portion (110) and the proximal portion (120). In some embodiments, the straight portion (132) is from 1 to 5 mm. In some embodiments, the straight portion (132) is from 0 to 2 mm. In some embodiments, the straight portion (132) is from 2 to 5 mm. In some embodiments, the straight portion (132) is from 5 to 7 mm. In some embodiments, the straight portion (132) is from 7 to 10 mm. In some embodiments, the straight portion (132) is more than 10 mm.

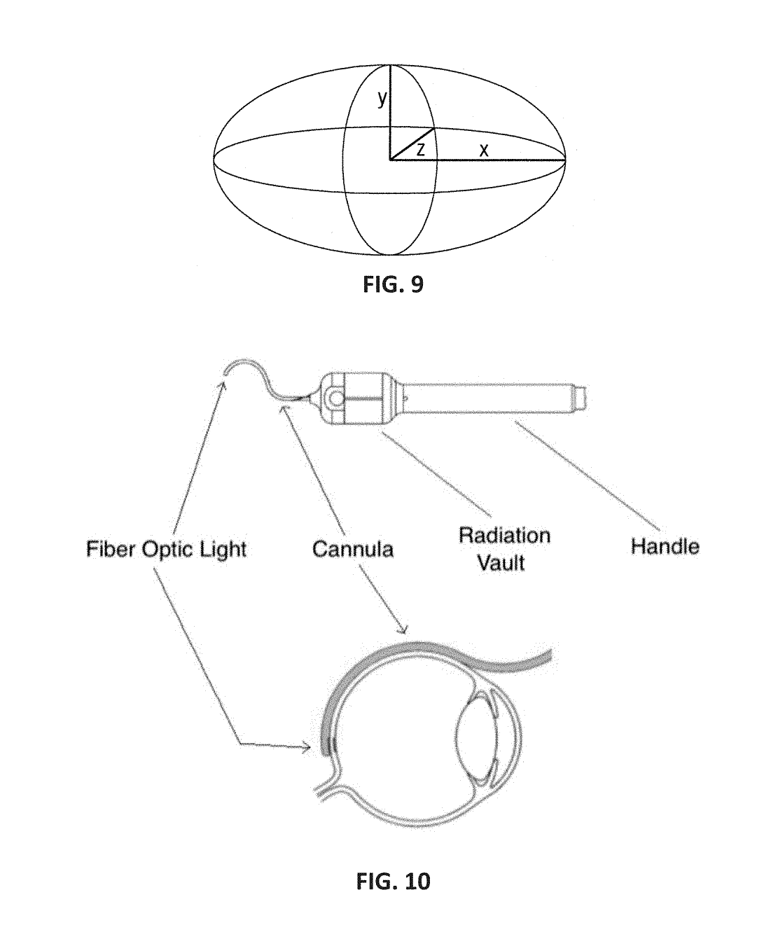

In some embodiments, the distal portion (110) has a shape of an arc formed from a connection between two points located on an ellipsoid, the ellipsoid having an x-axis dimension “a”, a y-axis dimension “b,” and a z-axis dimension “c.” FIG. 9 shows an example of an ellipsoid with the x-axis, y-axis, and z-axis. In some embodiments, “a” is from about 0 to 1 meter (e.g., from 0 to 50 mm, from 1 to 50 mm, from 5 to 50 mm, from 10 to 20 mm, from 10 to 40 mm, from 10 to 50 mm, from 15 to 50 mm, from 20 to 50 mm, from 30 to 50 mm, from 1 to 100 mm, from 10 to 100 mm, from 25 to 100 mm, from 50 to 100 mm, from 20 to 200 mm, from 50 to 200 mm, from 100 to 200 mm, from 20 to 500 mm, from 50 to 500 mm, etc.), “b” is from about 0 to 1 meter (e.g., from 0 to 50 mm, from 1 to 50 mm, from 5 to 50 mm, from 10 to 20 mm, from 10 to 40 mm, from 10 to 50 mm, from 15 to 50 mm, from 20 to 50 mm, from 30 to 50 mm, from 1 to 100 mm, from 10 to 100 mm, from 25 to 100 mm, from 50 to 100 mm, from 20 to 200 mm, from 50 to 200 mm, from 100 to 200 mm, from 20 to 500 mm, from 50 to 500 mm, etc.), and “c” is from about 0 to 1 meter (e.g., from 0 to 50 mm, from 1 to 50 mm, from 5 to 50 mm, from 10 to 20 mm, from 10 to 40 mm, from 10 to 50 mm, from 15 to 50 mm, from 20 to 50 mm, from 30 to 50 mm, from 1 to 100 mm, from 10 to 100 mm, from 25 to 100 mm, from 50 to 100 mm, from 20 to 200 mm, from 50 to 200 mm, from 100 to 200 mm, from 20 to 500 mm, from 50 to 500 mm, etc.).

In some embodiments, the proximal portion (120) has a shape of an arc formed from a connection between two points on an ellipsoid, the ellipsoid having an x-axis dimension “d”, a y-axis dimension “e,” and a z-axis dimension “f.” FIG. 9 shows an example of an ellipsoid with the x-axis, y-axis, and z-axis. In some embodiments, “d” is from about 0 to 1 meter (e.g., from 0 to 50 mm, from 1 to 50 mm, from 5 to 50 mm, from 10 to 20 mm, from 10 to 40 mm, from 10 to 50 mm, from 15 to 50 mm, from 20 to 50 mm, from 30 to 50 mm, from 1 to 100 mm, from 10 to 100 mm, from 25 to 100 mm, from 50 to 100 mm, from 20 to 200 mm, from 50 to 200 mm, from 100 to 200 mm, from 20 to 500 mm, from 50 to 500 mm, etc.), “e” is from about 0 to 1 meter (e.g., from 0 to 50 mm, from 1 to 50 mm, from 5 to 50 mm, from 10 to 20 mm, from 10 to 40 mm, from 10 to 50 mm, from 15 to 50 mm, from 20 to 50 mm, from 30 to 50 mm, from 1 to 100 mm, from 10 to 100 mm, from 25 to 100 mm, from 50 to 100 mm, from 20 to 200 mm, from 50 to 200 mm, from 100 to 200 mm, from 20 to 500 mm, from 50 to 500 mm, etc.), and “f” is from about 0 to 1 meter (e.g., from 0 to 50 mm, from 1 to 50 mm, from 5 to 50 mm, from 10 to 20 mm, from 10 to 40 mm, from 10 to 50 mm, from 15 to 50 mm, from 20 to 50 mm, from 30 to 50 mm, from 1 to 100 mm, from 10 to 100 mm, from 25 to 100 mm, from 50 to 100 mm, from 20 to 200 mm, from 50 to 200 mm, from 100 to 200 mm, from 20 to 500 mm, from 50 to 500 mm, etc.).

In some embodiments, “a,” “b,” “c,” “d,” “e,” or “f” have a dimension from 1 to 3 mm. In some embodiments, “a,” “b,” “c,” “d,” “e,” or “f” have a dimension from 1 to 5 mm. In some embodiments, “a,” “b,” “c,” “d,” “e,” or “f” have a dimension from 3 to 5 mm. In some embodiments, “a,” “b,” “c,” “d,” “e,” or “f” have a dimension from 5 to 8 mm. In some embodiments, “a,” “b,” “c,” “d,” “e,” or “f” have a dimension from 8 to 10 mm. In some embodiments, “a,” “b,” “c,” “d,” “e,” or “f” have a dimension from 10 to 12 mm. In some embodiments, “a,” “b,” “c,” “d,” “e,” or “f” have a dimension from 12 to 15 mm. In some embodiments, “a,” “b,” “c,” “d,” “e,” or “f” have a dimension from 15 to 18 mm. In some embodiments, “a,” “b,” “c,” “d,” “e,” or “f” have a dimension from 18 to 20 mm. In some embodiments, “a,” “b,” “c,” “d,” “e,” or “f” have a dimension from 20 to 25 mm. In some embodiments, “a,” “b,” “c,” “d,” “e,” or “f” have a dimension greater than 25 mm. In some embodiments, “a,” “b,” “c,” “d,” “e,” or “f” have a dimension greater than 50 mm. In some embodiments, “a,” “b,” “c,” “d,” “e,” or “f” have a dimension from 9 to 15 mm. In some embodiments, “a,” “b,” “c,” “d,” “e,” or “f” have a dimension from 11 to 17 mm. In some embodiments, “a,” “b,” “c,” “d,” “e,” or “f” have a dimension from 7 to 13 mm.

The ellipsoid may be a sphere, wherein “a” is equal to “b”, and “b” is equal to “c”. The ellipsoid may be a scalene ellipsoid (e.g., triaxial ellipsoid) wherein “a” is not equal to “b”, “b” is not equal to “c”, and “a” is not equal to “c”. In some embodiments, the ellipsoid is an oblate ellipsoid wherein “a” is equal to “b”, and both “a” and “b” are greater than “c”. In some embodiments, the ellipsoid is a prolate ellipsoid wherein “a” is equal to “b”, and both “a” and “b” are less than “c”. In some embodiments, “a” is about equal to “b” (e.g., for an emmetropic eye). In some embodiments, “a” is not equal to “b” (e.g., for an emmetropic eye). In some embodiments, “b” is about equal to “c”. In some embodiments, “b” is not equal to “c”. In some embodiments, “a” is about equal to “c”. In some embodiments, “a” is not equal to “c”. In some embodiments, “d” is about equal to “e”. In some embodiments, “d” is not equal to “e”. In some embodiments, “e” is about equal to “f”. In some embodiments, “e” is not equal to “f”. In some embodiments, “d” is about equal to “f”. In some embodiments, “d” is not equal to “f”.

In some embodiments, the ellipsoid is egg-shaped or a variation thereof.

In some embodiments, the system (100) (e.g., cannula (105)) or portions thereof (e.g., distal portion (110), proximal portion (120), etc.) is constructed from a material comprising stainless steel, gold, platinum, titanium, the like, or a combination thereof. In some embodiments, the system (100) (e.g., cannula (105)) or portions thereof (e.g., distal portion (110), proximal portion (120), etc.) is constructed from a material comprising stainless steel (e.g., including but not limited to surgical stainless steel). In some embodiments, the system (100) (e.g., cannula (105)) or portions thereof (e.g., distal portion (110), proximal portion (120), etc.) is constructed from a material comprising other conventional materials such as Teflon, other metals, metal alloys, polyethylene, polypropylene, other conventional plastics, ceramics, 3D printed materials, 2 part resins, composites, or combinations of the foregoing may also be used. For example, the distal portion (110) may be constructed from a material comprising a plastic. As another example, a part of the distal portion (110) may be constructed from a material comprising a plastic, and the remainder of the distal portion (110) and the proximal portion (120) may be constructed from a material comprising a metal.

### RBS Holder

The system (100) further comprises a radionuclide brachytherapy source (RBS) holder (230) for holding an RBS (180). The RBS holder (230) is directly or indirectly connected to the distal portion (110) of the cannula (105), e.g., at the tip of the distal portion (110) of the cannula (105). In some embodiments, the RBS holder (230) is directly or indirectly connected to the straight distal portion (136) (e.g., see FIG. 2A). The RBS holders (230) may be loaded (e.g., manually, automated) with the RBS prior to use. Or, in some embodiments, the RBS (180) is within the RBS holder (230) prior to attachment of the RBS holder (230) to the cannula (105). For example, the RBS holder (230) may be generally solid, wherein the RBS (180) is disposed therein (e.g., an example wherein the RBS (180) is not removed from the RBS holder (230) but instead the RBS holder (230) is removable from the cannula (105).

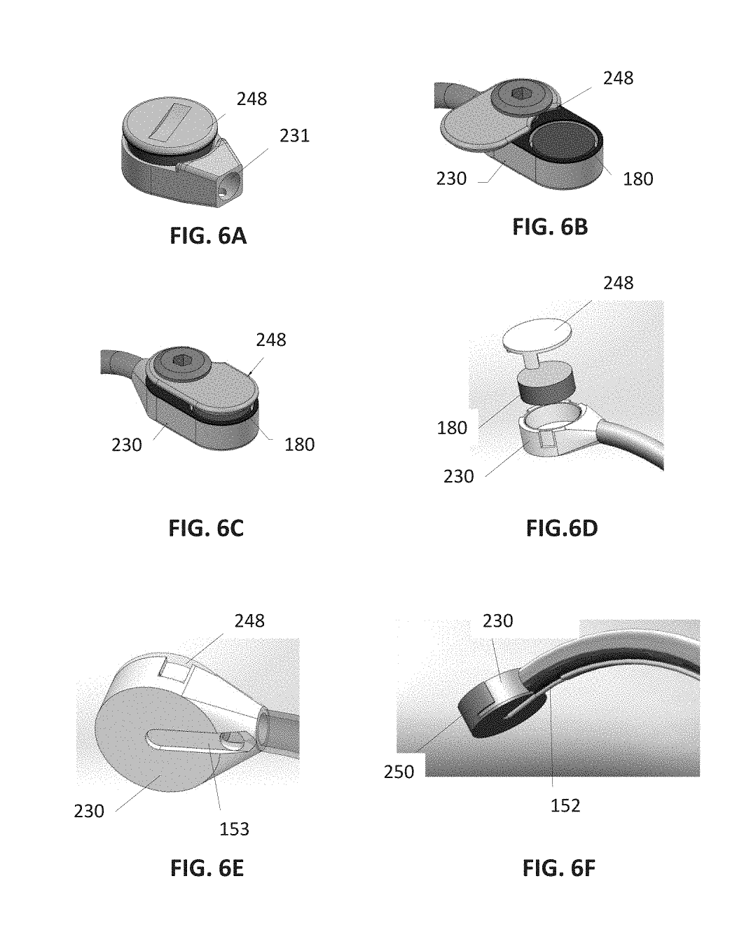

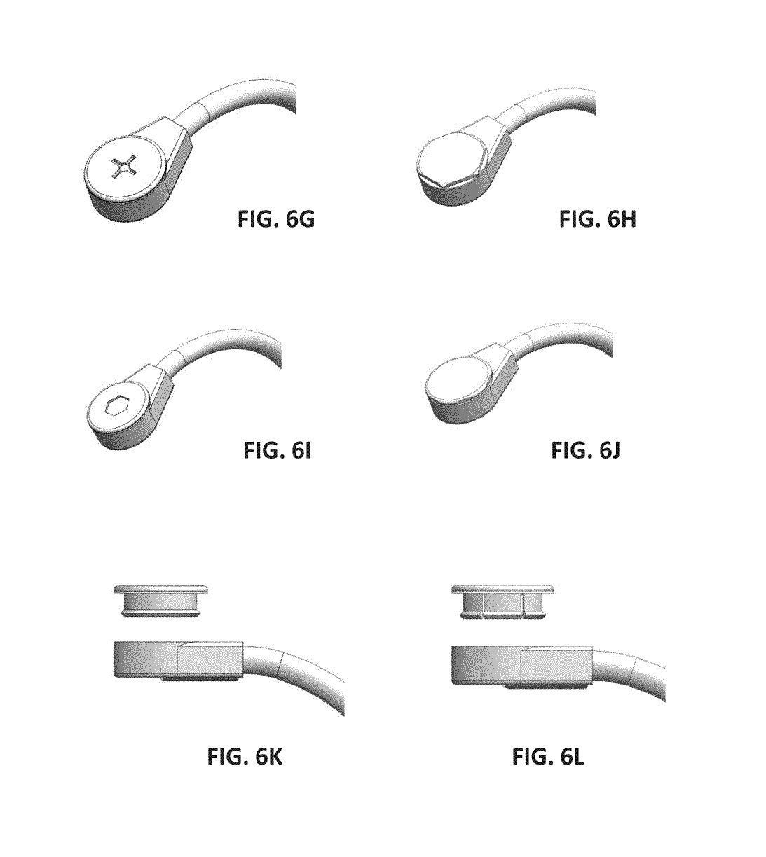

In some embodiments, the RBS holder (230) is in the shape of a disc (as shown in FIG. 6F) or teardrop (e.g., as shown in FIG. 2A). In some embodiments, the RBS holder (230) has a rounded tip (e.g., the tip being opposite the end attached to the cannula (105). The RBS holder (230) is not limited to this shape and may be constructed in any appropriate shape, e.g., ellipsoidal or oval, rectangular, etc.

The RBS holder (230) is adapted to accept an RBS (180). In some embodiments, the RBS is fixedly disposed in the RBS holder (230). In some embodiments, the RBS (180) is insertable and/or removable. The RBS may be inserted into a cavity (232) in the RBS holder (230). A cap (248) may be removably attachable to the RBS holder (230) in order to secure the RBS (180) in the RBS holder (230). The cap (248) may be adapted to seal the RBS (180) and cavity (232) from liquid. In some embodiments, a gasket is sandwiched between the cap (248) and RBS holder (230) to help seal the cavity (232). In some embodiments, the RBS holder may further comprise a centering device for the RBS.

In some embodiments, the cap (248) comprises a head configuration (249), e.g., a pattern of indentations in the top surface that allows for engagement with a screwdriver device or other appropriate device for removal of the cap (248). In some embodiments, the cap (248) does not have a head configuration (249) but instead has at least one exterior wall that can engage a socket of a wrench-like device. The head configuration shown in FIG. 1B comprises a central slot and a pair of opposing circular indentations. In some embodiments the cap (248) may be removed or inserted by engaging a device in the central slot. In some embodiments the cap (248) may be removed or inserted by engaging a device in the pair of opposing circular indentations (e.g. spanner screw driver). The present invention is not limited to this configuration and encompasses any head configuration that allows for opening and closing of the cap (248). In some embodiments, the cap (248) is a snap-on cap. In FIG. 1B, the cap (248) is disengaged from the RBS holder (230) and the cavity (232) is exposed.

The cap may utilize any number of common or uncommon ways to tighten and loosen such as but not limited to those shown herein, e.g., one or more slots, a spanner screw drive (e.g., two holes), an internal hex drive for a hex key, an external hex for a driver or socket, a cruciform type drive (e.g. Phillips screw drive feature), the like (e.g., square, hex, pentagon, external torx, 12-point, thumbscrew, slot, cross, Roberston, hex socket, hexalobular socket, double-square, combination drives, breakaway head, Bristol, clutch, claw, line, one-way, pentalobe, polydrive, etc.), or combinations thereof.

Other cap designs may be considered. For example, FIGS. 6A-L shows a variety of non-limiting examples of alternative cap (248) and RBS holder (230) designs. For example, FIG. 6A features only a single slot as the head configuration. FIG. 6B and FIG. 6C feature a pivot cap that can pivot (pivot about a pin or other component) in at least one direction to expose the cavity. FIG. 6D and FIG. 6E feature a snap-on cap. Note that the cap (248) in the example can snap from the outside as shown, or on the inside (e.g., as shown in FIG. 6K and FIG. 6L), e.g., using various configurations of caps and latches well known to one of ordinary skill in the art.

FIG. 6G shows a cap comprising a Phillips screw drive configuration. FIG. 6H shows a cap comprising an external hex head configuration. FIG. 6I of FIG. 6A shows a cap comprising an internal hex head drive configuration. FIG. 6J shows a cap comprising an external hex head configuration, e.g., a fine or slight version of an external hex (e.g., note the slight hex edges on the side edges of the cap). FIG. 6K shows an example of a snap-on cap, wherein the snap feature is inside the cap (in contrast to FIG. 6D wherein the snap feature of the snap-on cap is outside the cap). FIG. 6L shows another example of a snap-on cap with internal snap features, wherein reliefs are disposed in the snap feature. As previously discussed, the present invention is not limited to the configurations and components described herein or shown in FIGS. 6A-L.

In some embodiments, the cap may be destructively removed in any implementation if desired. In some embodiments, the cap may also be press fit and then destructively removed (e.g., cut off, etc.)

RBS holder (230) of the present invention may be constructed of any biocompatible material or a combination of materials. Examples, of biocompatible materials include, but are not limited to, metals (for example, stainless steel, titanium, gold), ceramics and polymers. In some embodiments, at least a portion of the RBS holder (230) is constructed from a material that shields the RBS. In some embodiments, the RBS holder (230) does not comprise material that shields the RBS. In some embodiments, the RBS holder further comprises shielding that may be used to shape the source profile, e.g., the RBS holder may comprise one or more masks or radiation shapers (e.g., placed in the holder) to shape the radiation profile delivered to target. In some embodiments, the cavity (232) may be constructed so as to shape the radiation profile. In some embodiments, a radiation shaper may be placed in the cavity (232) or outside the cavity (232) for shaping the radiation profile. In some embodiments, a radiation shaper may be placed outside the well (232) and attached using an attachment mechanism such as but not limited to a clip or adhesive.

### Straight Distal Portion

In some embodiments, a straight distal portion (136) is disposed at the end of the distal portion (110) or at the end of the kink (138) (the kink is described below). The straight distal portion (136), for example, may help the RBS holder (230) conform to curvature of the distal portion. In some embodiments, the straight distal portion (136) helps put the RBS holder (230) in an appropriate position for achieving proper placement around the globe of the eye, e.g., enables placement closer to the target.

In some embodiments, the straight distal portion (136) has a length from 0.001 mm to 0.01 mm, 0.001 mm to 0.1 mm, 0.001 mm to 1 mm, 0.001 mm to 5 mm, 0.001 mm to 10 mm, 0.001 mm to 25 mm, or 0.001 mm to greater than 25 mm, etc. In some embodiments, the straight distal portion (136) has a length from 0.01 mm to 0.1 mm, 0.01 mm to 1 mm, 0.01 mm to 5 mm, 0.01 mm to 10 mm, 0.01 mm to 25 mm, or 0.01 mm to greater than 25 mm, etc. In some embodiments, the straight distal portion (136) has a length from 0.1 mm to 1 mm, 0.1 mm to 5 mm, 0.1 mm to 10 mm, 0.1 mm to 25 mm, or 0.1 mm to greater than 25 mm, etc. The present invention is not limited to these ranges. For example, the straight distal portion (136) may be any appropriate length to adjust for positioning of the cannula (105) or the RBS holder (230).

In some embodiments, the end of the straight distal portion (136) connects (e.g., fixedly, removably) to the RBS holder (230). In some embodiments, the end of the cannula (105), e.g., the distal portion (110), the straight distal portion (136), etc., may connect to the cap (248). In some embodiments, the cavity may attach to the cap (248). In some embodiments, the straight distal portion (136) engages the socket (231) of the RBS holder (230). In some embodiments, the straight distal portion and RBS holder allow for the incorporation of a light system (150), e.g., incorporation of a fiber optic light.

### Distal Portion Kink

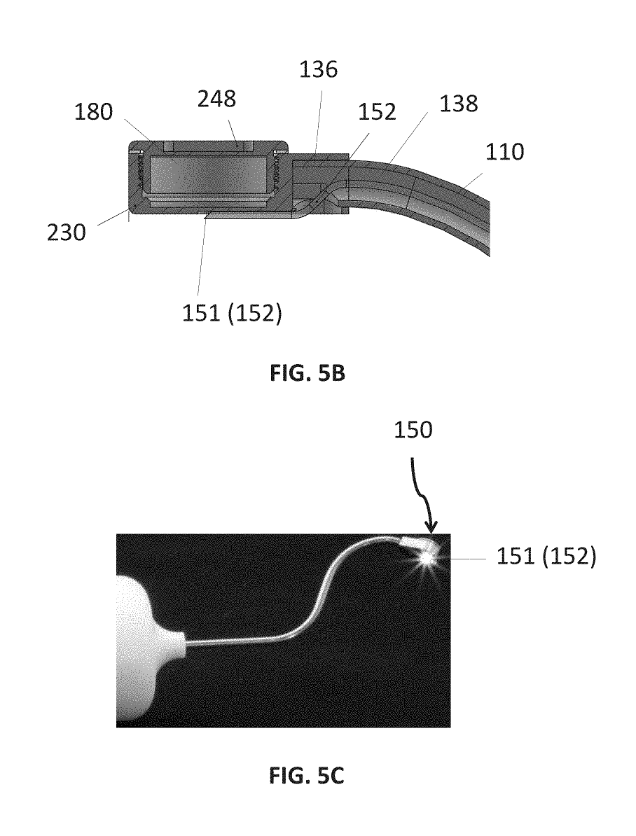

In some embodiments, a kink (138) is disposed at the end of the distal portion (110). The kink (138) may function to help align the RBS holder (230), e.g., to help align the cavity or RBS on the curve of the distal portion (110). In some embodiments, in the case of a straight RBS holder or straight cavity or straight RBS, the kink can help put those components in a position to be tangent to the distal curve. In some embodiments, a kink (138) is disposed in between the distal portion (110) and straight distal portion (136). The kink (138), for example, may help the RBS holder (230) conform to curvature of the distal portion (110). In some embodiments, the kink (138) helps put the RBS holder (230) in an appropriate position for achieving proper placement around the globe of the eye. Detailed views of the kink (138) are shown in FIG. 2A, FIG. 3, FIG. 5A, and FIG. 5B, wherein the kink (138) is shown between the distal portion (110) and straight distal portion (136).

The size and/or curvature of the kink (138) may be determined by where the RBS (or RBS holder or cavity, etc.) is to be positioned. For example, the curvature of the distal portion, the length of the straight distal portion, the size of the RBS holder may all factor into the size and/or shape or curve of the kink (138). Thus, the kink (138) may have any appropriate radius of curvature or arc length as necessary.

In some embodiments, the kink (138) has a radius of curvature from 5 to 8 mm (e.g., 5 mm, 6 mm, 6.5 mm, 6.75 mm, 7 mm, 8 mm, etc.). In some embodiments, the kink (138) has a radius of curvature from 3 to 10 mm (e.g., 3 mm, 4 mm, 5 mm, 6 mm, 7 mm, 8 mm, 9 mm, 10 mm, etc.). In some embodiments, the kink (138) has a radius of curvature from 1 to 20 mm. In some embodiments, the kink (138) has a radius of curvature from 1 to 30 mm. In some embodiments, the kink (138) has a radius of curvature from 4 to 40 mm. In some embodiments, the kink (138) has a radius of curvature from 1 to 50 mm. In some embodiments, the kink (138) has a radius of curvature from 5 to 50 mm. In some embodiments, the kink (138) has a radius of curvature from 1 to 100 mm. In some embodiments, the kink (138) has a radius of curvature from 1 to 200 mm. In some embodiments, the kink (138) has a radius of curvature from 1 to 500 mm. In some embodiments, the kink (138) has a radius of curvature greater than 500 mm, e.g., 600 mm, 700 mm, 800 mm, 900 mm, 1,000 mm (1 m), etc.

In some embodiments, the kink (138) has an arc length from 0.1 to 10 mm (e.g., 1 mm, 2 mm, 3 mm, 5 mm, 7 mm, 10 mm, etc.). In some embodiments, the kink (138) has an arc length from 1 to 5 mm (e.g., 2 mm, 3 mm, 5 mm, etc.). In some embodiments, the kink (138) has an arc length from 0.5 to 10 mm. In some embodiments, the kink (138) has an arc length from 0.5 to 20 mm. In some embodiments, the kink (138) has an arc length from 1 to 10 mm. In some embodiments, the kink (138) has an arc length from 1 to 20 mm. In some embodiments, the kink (138) has an arc length from 2 to 10 mm. In some embodiments, the kink (138) has an arc length from 2 to 20 mm. In some embodiments, the kink (138) has an arc length greater than 20 mm or less than 1 mm (e.g., 0.5 mm, 0.4 mm, 0.1 mm, 0.05, 0.01 etc.).

In some embodiments, the end of the kink (138) connects (e.g., fixedly, removably) to the RBS holder (230). In some embodiments, the kink (138) may connect to the cap (248). In some embodiments, the kink (138) engages the socket (231) of the RBS holder (230). In some embodiments, the kink (138) and/or RBS holder (230) and/or straight distal portion (136) allow for the incorporation and/or connection of a light system (150), e.g., incorporation of a fiber optic light.

### Handle

As previously discussed, in some embodiments, a straight proximal portion (134) and/or handle (140) may extend from the proximal portion (120). The handle (140) may be removably or fixedly attached to the cannula (105) (e.g., straight proximal portion (134), proximal portion (120), etc.). The handle (140) may be directly or indirectly attached to the cannula (105) (e.g., straight proximal portion (134), proximal portion (120), etc.). As shown in FIG. 1A, a straight proximal portion (134) connects the handle (140) (e.g., a distal end of the handle (140)) to the proximal portion (120).

In some embodiments, the distal portion of the handle (140) and/or proximal portion of the handle (140) and/or middle portion of the handle (140) have different sizes, e.g., widths, e.g., diameters, lengths, wall thickness if a hollow handle, etc. The handle (140) in FIG. 1A features a wider distal end of the handle (140) as compared to the middle area of the handle (140) and proximal end of the handle (140). The present invention is not limited to this configuration. For example, in some embodiments, the handle (140) is wider on both ends as compared to the middle. In some embodiments, the handle (140) has a generally similar width across its length. In some embodiments, the middle is wider than one or both of the ends.

In some embodiments, the handle (140) and/or cannula (105) comprises an alignment component (143), e.g., a marking or feature that allows a user to align one or more features of the system (100), e.g., the cannula (105), e.g., when treating a patient. For example, the alignment component (143) may help the user position the cannula in the appropriate place on the eye. In some embodiments, an alignment component (143) is disposed in or on the handle (140), e.g., the distal portion of the handle, the middle portion, the proximal portion, or combinations of the distal, middle, and proximal portions of the handle (140). In some embodiments, the alignment component (143) is disposed in or on the cannula (105). In some embodiments, the alignment component (143) or multiple alignment components may be disposed in or on the handle (140) and cannula (105).

In some embodiments, handle (140) comprises a gripping component (144) adapted to help a user hold onto the handle (140) more comfortably and/or securely. Gripping components are well known to one of ordinary skill in the art. For example, in some embodiments, the gripping component comprises grooves, as shown in FIG. 1A. In some embodiments, the gripping component comprises bumps, indentations or scratches, the like, or a combination thereof. In some embodiments, the handle (140) does not feature a gripping component (144).

In some embodiments, the alignment component (143) is aligned with the cannula (105), e.g., as shown in FIG. 1A (the axis of the cannula (105) is in line with that of the alignment component (143). In some embodiments, the alignment component (143) is arranged in a way that allows the user to know how the cannula (105) is positioned, e.g., the alignment component (143) allows for orientation of the cannula via visualization or tactile sensation. In some embodiments, the alignment component (143) is a mark or distinction (e.g., line, symbol, etc.) that is visualized. In some embodiments, the alignment component (143) is a physical feature that allows the user to sense alignment of the cannula by touch (tactile sensation). An alignment component (143) may, for example, comprise an indentation, a bump, or even a variation in the gripping component (144) of the handle (140). For example, as shown in FIG. 1A, the alignment component (143) is an inwardly curved indentation that creates a gap in the gripping component (144) (e.g., grooves). The alignment component (143) may be any appropriate mechanism or component that provides guidance to the user as to the alignment of the cannula.

In some embodiments, the alignment component (143) is anywhere on the handle (140). In some embodiments, the alignment component (143) may be on the cannula (105), the handle (140), or both the cannula (105) and handle (140). For example, the alignment component (143) may be a feature or a mark on the cannula itself.

In some embodiments, the cannula (105) and handle (140) are preassembled as one piece. In some embodiments, the cannula (105) and handle (140) are constructed as two or more pieces. In some embodiments, the cannula (105) and handle (140) are assembled prior to inserting into the eye. In some embodiments, the cannula (105) and handle (140) are assembled after inserting the cannula (105) into posterior portion of the eye according to the present invention.

### Radiation Shielding

In some embodiments, one or more components of the system (100) of the present invention are constructed from a material that can further shield the user from the RBS. For example, a side of the distal portion (110) opposite the side that contacts the sclera may be constructed from a material that can further shield the patient from the RBS. In some embodiments, a material having a low atomic number (Z) may be used for shielding (e.g., polymethyl methacrylate). In some embodiments, one or more layers of material are used for shielding, wherein an inner layer comprises a material having a low atomic number (e.g., polymethyl methacrylate) and an outer layer comprises lead.

In some embodiments, the inner layer (e.g., polymethyl methacrylate or other material) is about 1.0 mm thick and the outer layer (e.g., lead or other material) is about 0.16 mm thick. In some embodiments, the inner layer (e.g., polymethyl methacrylate or other material) is from 0.1 mm to 1.0 mm thick and the outer layer (e.g., lead or other material) is from 0.01 mm to 0.10 mm thick. In some embodiments, the inner layer (e.g., polymethyl methacrylate or other material) is from 0.1 mm to 1.0 mm thick and the outer layer (e.g., lead or other material) is from 0.10 mm to 0.20 mm thick. In some embodiments, the inner layer (e.g., polymethyl methacrylate or other material) is from 1.0 mm to 2.0 mm thick and the outer layer (e.g., lead or other material) is from 0.15 mm to 0.50 mm thick. In some embodiments, the inner layer (e.g., polymethyl methacrylate or other material) is from 2.0 mm to 5.0 mm thick and the outer layer (e.g., lead or other material) is from 0.25 mm to 1.0 mm thick.

In some embodiments, the system (100) further comprises a removable or movable shield for shielding (e.g., temporarily shielding) the RBS (180). In some embodiments, the shield attaches to (e.g., removably, slidably, etc.) the area of the system (100) that holds the RBS. For example, in some embodiments, a removable shield (250) can temporarily cover the RBS holder (180) (see FIG. 6F).

### Light Source on the Cannula

In some embodiments, the system (100) further comprises a light system (150), e.g., a light source, a light emitting component, etc. The light system (150) or components thereof (e.g., light source, light emitting component) may be disposed on the cannula (105) or RBS holder (230). Various different types of light systems may be considered. For example, in some embodiments, the light system (150) comprises a light source connected to a light emitting component (151). In some embodiments, the light emitting component (151) is the light source. As an example, in some embodiment, the light system (150) comprises a fiber optic light wire (152) that is connectable to a light source. The wire (152) may travel through at least a part of the brachytherapy system (100). The tip of the wire (152) may function as the light emitting component (151) since light is emitted from the tip.

In some embodiments, the light system (150) or a portion thereof (e.g., the tip of the fiber optic light wire (152) is disposed or housed in the RBS holder (230). In some embodiments, the light system (150) extends from the RBS holder (230) through at least a portion of the cannula (105), e.g., the fiber optic light wire (152), may extend through at least a portion of the cannula (105). In some embodiments, the light system (150) or a portion thereof (e.g., the fiber optic light wire (152)) extends from the RBS holder (230) through the cannula (105) and through at least a portion of the handle (140). FIG. 1A shows the fiber optic wire (152) extending from the end of the handle (140). FIG. 5A, FIG. 5B, and FIG. 5C show detailed views of the light system (150) configuration of FIG. 1A, wherein the tip of the fiber optic light wire (152) (the tip being the light emitting component (151)) is engaged in a slot (153) in the bottom surface (230b) of the RBS holder (230). In some embodiments, the tip being the light emitting component (151/152) is positioned in the center of the RBS holder and/or RBS source positioned in the RBS holder. In some embodiments, the tip being the light emitting component (151/152) is positioned off -center relative to the RBS source.

The present invention is not limited to fiber optic wires. For example, in some embodiments, the light system (150) comprises a light emitting diode (LED) or other light. The LED may be housed in the RBS holder. The LED may be operatively connected to a battery or other power source housed within a portion of the system (100) (e.g., in the RBS holder (180)) or external to the system (100).

Any other appropriate light system may be used. For example, in some embodiments, the light system (150) features an LED with wires routed in the cannula (105) and/or outside the cannula. Wires may run to an on-board battery or external power source. The LED power source may be located in the handle or elsewhere, e.g., there may be a battery pack in the handle. The battery pack may be one use or rechargeable. In some embodiments, the light system (150) features a fiber optic system with an external light source and/or power source. In some embodiments, the light system (150) features a fiber optic system with a light source contained within the handle and/or cannula, e.g., either battery powered or externally powered (or both). Any self powered lighting may be considered, such as but not limited to tritium and a phosphor.

The light emitted from the light system (150) may be seen through transillumination and may help guide the surgeon to the correct positioning of the system (100).

In some embodiments, the light system (150) illuminates the target area. In some embodiments, the light system (150) illuminates a portion of the target area. In some embodiments, the light system (150) illuminates the target area and a non-target area. As used herein, a “target area” is the area receiving about 100% of the intended therapeutic radiation dose. In some embodiments, light system (150) illuminates more than the targeted radiation zone. The light from the light system (150) may extend beyond the lesion to make reference points (e.g., optic nerve, fovea, vessels) visible which may help orient the user (e.g., physician, surgeon).

In some embodiments, the RBS holder (230) is not fixedly attached to the cannula (105) but is attachable, e.g., prior to insertion in a patient (e.g., if a system featured incorporation or insertion of an RBS in an RBS holder (230) prior to attaching the RBS holder (230) to the cannula (105). In some embodiments, the light system (150) can be connected when the RBS holder (230) is attached to the cannula (105), e.g., the distal portion (110), the straight distal portion (136). For example, if fiber optic light wires were used, the wires would connect when the RBS holder (230) is attached to allow light to pass from the light source through the wires to the tip of the wire.

In some embodiments, the light system (150), e.g., the fiber optic wires, can be autoclaved. In some embodiments, the fiber optic wires are glass.

In some embodiments, the system (100) comprises luminescent paint. For example, in some embodiments, luminescent paint is disposed on a portion of the RBS holder (230) and/or cannula (150), wherein the luminescent paint may glow when activated. For example, the luminescent paint may glow when exposed to radiation (which may be used as an indication of the presence of the radiation).

### Radionuclide Brachytherapy Source

According to the Federal Code of Regulations, a radionuclide brachytherapy source (RBS) comprises a radionuclide encased in an encapsulation layer. For example, the Federal Code of Regulations defines a radionuclide brachytherapy source as follows: “A radionuclide brachytherapy source is a device that consists of a radionuclide which may be enclosed in a sealed container made of gold, titanium, stainless steel, or platinum and intended for medical purposes to be placed onto a body surface or into a body cavity or tissue as a source of nuclear radiation for therapy.”

The system (100) of the present invention may further comprise an RBS (180). The RBS (180) is adapted to irradiate a target, and the target receives a dose rate. In some embodiments, the target receives a dose rate of greater than about 10 Gy/min. In some embodiments, the RBS provides a dose rate of greater than about 11 Gy/min to the target. In some embodiments, the RBS provides a dose rate of greater than about 12 Gy/min to the target. In some embodiments, the RBS provides a dose rate of greater than about 13 Gy/min to the target. In some embodiments, the RBS provides a dose rate of greater than about 14 Gy/min to the target. In some embodiments, the RBS provides a dose rate of greater than about 15 Gy/min to the target. In some embodiments, the RBS provides a dose rate from 10 to 15 Gy/min. In some embodiments, the RBS provides a dose rate from 15 to 20 Gy/min. In some embodiments, the RBS provides a dose rate from 20 to 30 Gy/min. In some embodiments, the RBS provides a dose rate from 30 to 40 Gy/min. In some embodiments, the RBS provides a dose rate from 40 to 50 Gy/min. In some embodiments, the RBS provides a dose rate from 50 to 60 Gy/min. In some embodiments, the RBS provides a dose rate from 60 to 70 Gy/min. In some embodiments, the RBS provides a dose rate from 70 to 80 Gy/min. In some embodiments, the RBS provides a dose rate from 80 to 90 Gy/min. In some embodiments, the RBS provides a dose rate from 90 to 100 Gy/min. In some embodiments, the RBS provides a dose rate of greater than 100 Gy/min.

In some embodiments, the RBS provides a dose rate from 15 to 20 Gy/min to the target. In some embodiments, the RBS provides a dose rate from 20 to 25 Gy/min to the target. In some embodiments, the RBS provides a dose rate from 25 to 30 Gy/min to the target. In some embodiments, the RBS provides a dose rate from 30 to 35 Gy/min to the target. In some embodiments, the RBS provides a dose rate from 35 to 40 Gy/min to the target. In some embodiments, the RBS provides a dose rate from 40 to 50 Gy/min to the target. In some embodiments, the RBS provides a dose rate from 50 to 60 Gy/min to the target. In some embodiments, the RBS provides a dose rate from 60 to 70 Gy/min to the target. In some embodiments, the RBS provides a dose rate from 70 to 80 Gy/min to the target. In some embodiments, the RBS provides a dose rate from 80 to 90 Gy/min to the target. In some embodiments, the RBS provides a dose rate from 90 to 100 Gy/min to the target. In some embodiments, the RBS provides a dose rate greater than about 100 Gy/min to the target. In some embodiments, a dose of about 16 Gy is delivered to the target. In some embodiments, a dose of about 16 Gy to 20 Gy is delivered to the target. In some embodiments, a dose of about 20 Gy is delivered to the target. In some embodiments, a dose of about 24 Gy is delivered to the target. In some embodiments, a dose of about 20 Gy to 24 Gy is delivered to the target. In some embodiments, a dose of about 30 Gy is delivered to the target. In some embodiments, about 24 Gy to 30 Gy is delivered to the target. In some embodiments, a dose of about 30 Gy to 50 Gy is delivered to the target. In some embodiments, a dose of about 50 Gy to 100 Gy is delivered to the target. In some embodiments, a dose of about 75 Gy is delivered to the target.

In some embodiments, the system (100) is pre-loaded with RBS (180) prior to insertion of the cannula (150) into the patient. In some embodiments, the system (100) is after-loaded with radiation, e.g., the RBS (180), e.g., the RBS is moved to a treatment position after insertion of the cannula.

The RBS of the present invention is constructed in a manner that is consistent with the Federal Code of Regulations, but is not limited to the terms mentioned in the Code. For example, the RBS of the present invention may optionally further comprise a substrate (discussed below). Also, for example, in addition to being enclosed by the mentioned “gold, titanium, stainless steel, or platinum”, in some embodiments the radionuclide (isotope) of the present invention may be enclosed by a combination of one or more of “gold, titanium, stainless steel, or platinum”. In some embodiments, the radionuclide (isotope) of the present invention may be enclosed by one or more layers of an inert material comprising silver, gold, titanium, stainless steel, platinum, tin, zinc, nickel, copper, other metals, ceramics, glass, or a combination of these.

The RBS may be constructed in a variety of ways, e.g., the RBS may be constructed fixedly attached (e.g., made permanently) into the well or cap, e.g., using each piece as the capsule, by affixing it after encapsulation, etc. The RBS may be affixed directly to the cannula (105), e.g., and removable or non-removable. The RBS may be constructed in a number of ways, having a variety of designs and/or shapes and/or distributions of radiation. In some embodiments, the RBS comprises a substrate, a radioactive isotope (e.g., Strontium-90), and an encapsulation. In some embodiments, the isotope is coated on the substrate, and both the substrate and isotope are further coated with the encapsulation. In some embodiments, the radioactive isotope is embedded in the substrate. In some embodiments, the radioactive isotope is part of the substrate matrix. In some embodiments, the encapsulation may be coated onto the isotope, and optionally, a portion of the substrate. In some embodiments, the encapsulation is coated around the entire substrate and the isotope. In some embodiments, the encapsulation encloses the isotope. In some embodiments, the encapsulation encloses the entire substrate and the isotope. In some embodiments, the radioactive isotope is an independent piece and is sandwiched between the encapsulation and the substrate.

The RBS is designed to provide a controlled projection of radiation in a rotationally symmetrical (e.g., circularly symmetrical) shape onto the target. In some embodiments, the RBS has an exposure surface that has a rotationally symmetrical shape to provide for the projection of a rotationally symmetrical irradiation onto the target.

A shape having n sides is considered to have n-fold rotational symmetry if n rotations each of a magnitude of 360°/n produce an identical figure. In some embodiments, shapes described herein as being rotationally symmetrical are shapes having n-fold rotational symmetry, wherein n is a positive integer of 3 or greater.

In some embodiments, the rotationally symmetrical shape has at least 5-fold rotational symmetry (n=5). In some embodiments, the rotationally symmetrical shape has at least 6-fold rotational symmetry (n=6). In some embodiments, the rotationally symmetrical shape has at least 7-fold rotational symmetry (n=7). In some embodiments, the rotationally symmetrical shape has at least 8-fold rotational symmetry (n=8). In some embodiments, the rotationally symmetrical shape has at least 9-fold rotational symmetry (n=9). In some embodiments, the rotationally symmetrical shape has at least 10-fold rotational symmetry (n=10). In some embodiments, the rotationally symmetrical shape has infinite-fold rotational symmetry (n=∞). Examples of rotationally symmetrical shapes include but are not limited to a circle, a square, an equilateral triangle, a hexagon, an octagon, a six-pointed star, and a twelve-pointed star.

Without wishing to limit the present invention to any theory or mechanism, it is believed that the rotationally symmetrical geometry provides a fast fall off at the target periphery. In some embodiments, the rotationally symmetrical geometry provides a uniform fall off of radiation at the target periphery. In some embodiments, the fast fall off of radiation at the target periphery reduces the volume and/or area irradiated.

In some embodiments, a surface on the substrate is shaped in a manner to provide a controlled projection of radiation in a rotationally symmetrical shape onto the target. For example, in some embodiments, the bottom surface of the substrate is rotationally symmetrical, e.g., circular, hexagonal, octagonal, decagonal, and/or the like. When the radioactive isotope is coated onto such rotationally symmetrical bottom surface of the substrate a rotationally symmetrical exposure surface is created.

In some embodiments, the substrate is a disk, for example a disk having a height and a diameter. In some embodiments, the height of the disk is from 0.1 mm and 10 mm. For example, in some embodiments, the height of the disk is from 0.1 to 0.2 mm. In some embodiments, the height of the disk is from 0.2 to 2 mm, such as 1.5 mm. In some embodiments, the height of the disk is from 2 to 5 mm. In some embodiments, the height of the disk is from 5 to 10 mm. In some embodiments, the diameter of the disk is from 0.1 to 0.5 mm. In some embodiments, the diameter of the disk is from 0.5 to 10 mm. For example, in some embodiments, the diameter of the disk is from 0.5 to 2.5 mm, such as 2 mm. In some embodiments, the diameter of the disk is from 2.5 to 5 mm. In some embodiments, the diameter of the disk is from 5 to 10 mm. In some embodiments, the diameter of the disk is from 10 to 20 mm.

The substrate may be constructed from a variety of materials. For example, in some embodiments the substrate is constructed from a material comprising, a silver, an aluminum, a stainless steel, tungsten, nickel, tin, zirconium, zinc, copper, a metallic material, a ceramic material, a ceramic matrix, the like, or a combination thereof. In some embodiments, the substrate functions to shield a portion of the radiation emitted from the isotope. For example, in some embodiments, the substrate has thickness such that the radiation from the isotope cannot pass through the substrate. In some embodiments, the density times the thickness of the substrate is from 0.01 g/cm² to 10 g/cm².

The substrate may be constructed in a variety of shapes. For example, the shape may include but is not limited to a cube, a sphere, a cylinder, a rectangular prism, a triangular prism, a pyramid, a cone, a truncated cone, a hemisphere, an ellipsoid, an irregular shape, the like, or a combination of shapes. In some embodiments, the substrate may have a generally rectangular side cross section. In some embodiments, the substrate may have a generally triangular or trapezoidal side cross-section. In some embodiments, the substrate may have generally circular/oval side cross section. The side cross section of the substrate 361 may be a combination of various geometrical and/or irregular shapes. Further, the present invention includes a combination of any of the shapes, e.g., two discs in one well, a disc and a square, four discs and an ellipse, or any other appropriate combination. In some embodiments, the combination of shapes are stacked or side by side or in any three dimensional combination so as to achieve a desired treatment zone in three dimensional space.

In some embodiments, the isotope is coated on the entire substrate. In some embodiments, the isotope is coated or embedded on a portion of the substrate (e.g., on the bottom surface of the substrate) in various shapes. For example, the coating of the isotope on the substrate may be in the shape of a rotationally symmetrical shape, e.g., a circle, a hexagon, an octagon, a decagon, or the like. The rotationally symmetrical shape of the isotope coating on the bottom surface of the substrate provides for the rotationally symmetrical exposure surface, which results in a controlled projection of radiation in a rotationally symmetrical shape onto the target.

In some embodiments, the encapsulation is constructed to provide a rotationally symmetrical exposure surface for a controlled projection of radiation having a rotationally symmetrical shape on the target. In some embodiments, the encapsulation has variable thickness so that it shields substantially all of the radiation in some portions and transmits substantially all of the radiation in other portions. For example, in one embodiment, the density times the thickness of the encapsulation is 1 g/cm² at distances greater than 1mm from the center of the radioactive portion of the source and the density times the thickness of the encapsulation is 0.01 g/cm² at distances less than 1 mm from the center of the radioactive portion of the source. For a Sr-90 source, this encapsulation would block substantially all of the radiation emitted more than 1 mm from the center of the radioactive portion of the source, yet permit substantially all of the radiation emitted within 1 mm of the center of the radioactive portion of the source to pass through. In some embodiments, the thickness of the encapsulation varies between 0.001 g/cm² and 10 g /cm². In some embodiments, rotationally symmetric shapes of the high and low density regions as described above are used.

The encapsulation may be constructed from a variety of materials, for example from one or more layers of an inert material comprising a steel, a silver, a gold, a titanium, a platinum, another bio-compatible material, the like, or a combination thereof. In some embodiments, the encapsulation is about 0.01 mm thick. In some embodiments, the encapsulation is from 0.01 to 0.10 mm thick. In some embodiments, the encapsulation is from 0.10 to 0.50 mm thick. In some embodiments, the encapsulation is from 0.50 to 1.0 mm thick. In some embodiments, the encapsulation is from 1.0 to 2.0 mm thick. In some embodiments, the encapsulation is more than about 2.0 mm thick, for example about 3, mm, about 4 mm, or about 5 mm thick. In some embodiments, the encapsulation is more than about 5 mm thick, for example, 6 mm, 7 mm, 8 mm, 9 mm, or 10 mm thick.

In some embodiments, a radiation-shaper can provide a controlled projection of radiation in a rotationally symmetrical shape onto the target. A radiation-shaper comprises a radio-opaque portion and a substantially radioactive transparent portion (hereinafter “window”). In some embodiments, the radiation shaper is placed under the RBS. The radiation from the portion of the RBS that overlaps the window is emitted through the window toward the target, and the radiation from the portion that does not overlap the window is blocked by the radio-opaque portion from reaching the target. Thus, a window having a rotationally symmetrical shape will allow for a projection of a rotationally symmetrical irradiation of the target.

As discussed, a controlled projection of radiation in a rotationally symmetrical shape onto the target allows for a fast fall off at the edge of the target. Also intended to be within the scope of the present invention are the various combinations of arrangements of the components of the RBS and/or cannula to produce a controlled projection of radiation in a rotationally symmetrical shape onto a target. Based on the disclosures herein, one of ordinary skill would know how to develop these various combinations to produce a controlled projection of radiation in a rotationally symmetrical shape onto the target allows for a fast fall off at the edge of the target. Fast fall off at the edge of the target may also be enhanced by recessing the RBS in a well having deep radio opaque walls.

### Isotopes & Radioactivity

Various isotopes may be employed within the scope of the present invention. Beta emitters such as phosphorus 32 and strontium 90 were previously identified as being useful radioactive isotopes because they are beta emitters that have limited penetration and are easily shielded. In some embodiments, the isotope comprises phosphorus 32 (P-32), strontium-90 (Sr-90), ruthenium 106 (Ru-106), yttrium 90 (Y-90), the like, or a combination thereof.

Although they are distinctly different from beta emitters, in some embodiments, the RBS may comprise an isotope such as a gamma emitter and/or an alpha emitter. For example, in some embodiments, the isotope comprises iodine 125 (I-125), palladium 103 (Pd-103), cesium 131 (Cs-131), cesium 137 (Cs-137), cobalt 60 (co-60), the like, or a combination thereof. In some embodiments, the RBS comprises a combination of various types of isotopes. For example, in some embodiments, the isotope comprises a combination of Sr-90 and P-32. In some embodiments, the isotope comprises a combination of Sr-90 and Y-90.

To achieve a particular dose rate at the target, the activity of the isotope that is to be used is determined for a given distance between the isotope and the target. For example, if the radiation source is a strontium-yittrium-90 titanate internally contained in a silver-clad matrix forming a disk about 4 mm in diameter and having a height of about 0.06 mm, sealed in titantium that is about 0.8 mm thick on one flat surface of the disk and around the circumference and is about 0.1 mm thick on the opposite flat surface of the disk (target side of the disk), the target is at a depth of about 1.5 mm (in tissue) and the desired dose rate is about 24 Gy/min at the target, an activity of about 100 mCi may be used. Or, if all aspects of the source are kept the same except that the diameter of the strontium-yittrium-90 titanate internally contained in a silver-clad matrix disk is about 3 mm in diameter, the target is at a depth of about 2.0 mm (in tissue) and the desired dose rate is about 18 Gy/min at the target, an activity of about 150 mCi may be used. Or, if all aspects of the source are kept the same except that the diameter of the strontium-yittrium-90 titanate internally contained in a silver-clad matrix disk is about 3 mm in diameter, the target is at a depth of about 0.5 mm (in tissue) and the desired dose rate is about 15 Gy/min at the target, an activity of about 33 mCi may be used. Or, if all aspects of the source are kept the same except that the diameter of the strontium-yittrium-90 titanate internally contained in a silver-clad matrix disk is about 2 mm in diameter, the target is at a depth of about 5.0 mm (in tissue) and the desired dose rate is about 30 Gy/min at the target, an activity of about 7100 mCi may be used.

In some embodiments, the isotope has about 5 to 20 mCi, for example, 10 mCi.

In some embodiments, to achieve a particular dose rate at the target, the radioactivity of the isotope that is to be used is determined for a given distance between the isotope and the target. For example, if the Sr-90 isotope is about 5 mm from the target (in tissue) and the desired dose rate is about 20 Gy/min at the target, a Sr-90 isotope having a radioactivity of about 5,000 mCi may be used. Or, if the P-32 isotope is about 2 mm from the target and the desired dose rate is about 25 Gy/min at the target, a P-32 isotope having a radioactivity of about 333 mCi may be used.

In some embodiments, the isotope has an activity of from 0.5 to 5 mCi. In some embodiments, the isotope has an activity of from 5 to 10 mCi. In some embodiments, the isotope has an activity of from 10 to 50 mCi. In some embodiments, the isotope has an activity of from 50 to 100 mCi. In some embodiments, the isotope has an activity of from 100 to 500 mCi. In some embodiments, the isotope has an activity of from 500 to 1,000 mCi. In some embodiments, the isotope has an activity of from 1,000 to 5,000 mCi. In some embodiments, the isotope has an activity of from 5,000 to 10,000 mCi. In some embodiments, the isotope has an activity of more than about 10,000 mCi.

Without wishing to limit the present invention to any theory or mechanism, it is believed that an effective design for a medical device for treating wet age-related macular degeneration may have a radiation dose distribution such that greater than 1 of the total source radiation energy flux (e.g., total radiation energy flux at the source center along the line l_{R}) is transmitted to greater than or equal to 1 cm distance from the RBS (along the line l_{R}). In some embodiments, the present invention has a RBS that deposits less than about 99% (e.g., 98%, 97%, etc.) of its total source radiation energy flux at distance of 1 cm or less from the RBS. In some embodiments, the present invention has a RBS that deposits more than 1% (e.g., 2%, 3%, 4% etc.) of its total source radiation energy flux at distance of 1 cm or more from the RBS. In some embodiments, the present invention has a RBS that deposits between 1% to 15% of its total source radiation energy flux at distance of 1 cm or more from the RBS.

In some embodiments, the interaction of the isotope radiation (e.g., beta radiation) with the encapsulation (e.g., gold, titanium, stainless steel, platinum) converts some of the beta radiation energy to an emission of bremsstrahlung x-rays. These x-rays may contribute to the entire radiotherapy dose both in the prescribed target area and also penetrate further than beta radiation. Thus such a device as constructed with the aforementioned desirable attributes with a primary beta source will produce a radiation pattern in which 1% or greater of all radiation from the source is absorbed at a distance greater than 1 cm (e.g., the radiation energy flux at a distance of 1 cm away from the center of the target is greater than 1% of the total source radiation energy flux).

In some embodiments, the present invention features a device wherein the RBS comprises an isotope, wherein the isotope comprises a beta radiation isotope, wherein about 1% of the total source radiation energy flux falls at a distance greater than 1 cm from the center of the target.

Without wishing to limit the present invention to any theory or mechanism, it is believed that it may be desirable to construct the RBS as described in the present invention for ease of manufacturing and so it is inert to the body (due to encasing the RBS in a bio-compatible material). A RBS that is constructed in this manner may produce a radiation pattern comprising beta rays, x-rays, or both beta rays and x-rays, such that greater than 1% of the total source radiation energy flux will extend a distance greater that about 1 cm.

In some embodiments, the RBS is in the form of a deployable wafer. In some embodiments, the wafer is in the shape of a cylinder, an ellipse, or the like. In some embodiments, the wafer comprises a nickel titanium (NiTi) substrate, either doped with or surface coated with an isotope and then encapsulated, that opens up when deployed. In some embodiments, the wafer is encapsulated with a bio-inert material if it is to be left in place for an extended period of time.

As used herein, the term “lateral” and/or “laterally” refers to in the direction of any line that is perpendicular to line l_{R}, wherein line l_{R} is the line derived from connecting the points l_{S} and l_{T}, wherein l_{S} is the point located at the center of the RBS and l_{T} is the point located at the center of the target. As used herein, the term “forwardly” refers to in the direction of and/or along line l_{R} from l_{S} through l_{T}.

As used herein, the term “substantially uniform” refers to a group of values (e.g., two or more values) wherein each value in the group is no less than about 90% of the highest value in the group. For example, an embodiment wherein the radiation doses at a distance of up to about 1 mm from the center of the target are substantially uniform implies that any radiation dose within the distance of up to about 1 mm away from the center of the target is no less than about 90% of the highest radiation dose within that area (e.g., the total target center radiation dose). For example, if a group of relative radiation doses within a distance of up to about 1 mm away from the center of the target are measured to be 99, 97, 94, 100, 92, 92, and 91, the relative radiation doses are substantially uniform because each value in the group is no less than 90% of the highest value in the group (100).

As used herein, the term “isodose” (or prescription isodose, or therapeutic isodose) refers to the area directly surrounding the center of the target wherein the radiation dose is substantially uniform.

Without wishing to limit the present invention to any theory or mechanism, the devices and methods of the present invention are believed to be effective by delivering a substantially uniform dose to the entire target region (e.g., neovascular tissue), or a non-uniform dose, in which the center of the target has dose that is about 2.5× higher than the dose at the boundary regions of the target.

### Dose Rates

The medical radiation community believes as medico-legal fact that low dose rate irradiation (e.g., less than about 10 Gy/min) is preferred over high dose rate irradiation because high dose rate irradiation may cause more complications. For example, the scientific publication “Posttreatment Visual Acuity in Patients Treated with Episcleral Plaque Therapy for Choroidal Melanomas: Dose and Dose Rate Effects” (Jones, R., Gore, E., Mieler, W., Murray, K., Gillin, M., Albano, K., Erickson, B., International Journal of Radiation Oncology Biology Physics, Volume 52, Number 4, pp. 989-995, 2002) reported the result “macula dose rates of 111 cGy/h (+/−11.1 cGy/h) were associated with a 50% risk of significant visual loss,” leading them to conclude “higher dose rates to the macula correlated strongly with poorer posttreatment visual outcome.” Furthermore, the American Brachytherapy Society (ABS) issued their recommendations in the scientific publication, “The American Brachytherapy Society Recommendations for Brachytherapy of Uveal Melanomas” (Nag, S., Quivey, J. M., Earle, J. D., Followill, D., Fontanesi, J., and Finger, P. T., International Journal of Radiation Oncology Biology Physics, Volume 56, Number 2, pp. 544-555, 2003) stating “the ABS recommends a minimum tumor 1-125 dose of 85 Gy at a dose rate of 0.60 to 1.05 Gy/h using AAPM TG-43 formalism for the calculation of dose.” Thus, the medical standard of care requires low dose rates.

Despite the teachings away from the use of high dose rates, the inventors of the present invention surprisingly discovered that a high dose rate (i.e., above about 10 Gy/min) may be advantageously used to treat neovascular conditions.

In some embodiments, the dose rate delivered/measured at the target is greater than 10 Gy/min (e.g., about 15 Gy/min, 20 Gy/min). In some embodiments, the dose rate delivered/measured at the target is from 10 Gy/min to 15 Gy/min. In some embodiments, the dose rate delivered/measured at the target is from 15 Gy/min to 20 Gy/min. In some embodiments, the dose rate delivered/measured at the target is from 20 Gy/min to 30 Gy/min. In some embodiments, the dose rate delivered/measured at the target is from 30 Gy/min and 40 Gy/min. In some embodiments, the dose rate delivered/measured at the target is from 40 Gy/min to 50 Gy/min. In some embodiments, the dose rate delivered/measured at the target is from 50 Gy/min to 75 Gy/min. In some embodiments, the dose rate delivered/measured at the target is from 75 Gy/min to 100 Gy/min. In some embodiments, the dose rate delivered/measured at the target is greater than about 100 Gy/min.

In some embodiments, about 16 Gy of radiation is delivered with a dose rate of about 16 Gy/min for about 1 minute (as measured at the target). In some embodiments, about 20 Gy of radiation is delivered with a dose rate of about 20 Gy/min for about 1 minute (as measured at the target). In some embodiments, about 25 Gy is delivered with a dose rate of about 12 Gy/min for about 2 minutes (as measured at the target). In some embodiments, about 30 Gy of radiation is delivered with a dose rate of greater than about 10 Gy/min (e.g., 11 Gy/min) for about 3 minutes (as measured at the target). In some embodiments, about 30 Gy of radiation is delivered with a dose rate of about 15 Gy/min to 16 Gy/min for about 2 minutes (as measured at the target). In some embodiments, about 30 Gy of radiation is delivered with a dose rate of about 30 Gy/min for about 1 minute (as measured at the target). In some embodiments, about 40 Gy of radiation is delivered with a dose rate of about 20 Gy/min for about 2 minutes (as measured at the target). In some embodiments, about 40 Gy of radiation is delivered with a dose rate of about 40 Gy/min for about 1 minute (as measured at the target). In some embodiments, about 40 Gy of radiation is delivered with a dose rate of about 50 Gy/min for about 48 seconds (as measured at the target). In some embodiments, about 50 Gy of radiation is delivered with a dose rate of about 25 Gy/min for about 2 minutes (as measured at the target). In some embodiments, about 50 Gy of radiation is delivered with a dose rate of about 75 Gy/min for about 40 seconds (as measured at the target). In some embodiments, a dose rate of about 75 Gy is delivered with a dose rate of about 75 Gy/min for about 1 minute (as measured at the target). In some embodiments, a dose rate of about 75 Gy is delivered with a dose rate of about 25 Gy/min for about 3 minutes (as measured at the target).

In some embodiments, the target is exposed to the radiation from 0.01 seconds to about 0.10 seconds. In some embodiments, the target is exposed to the radiation from 0.10 seconds to about 1.0 second. In some embodiments, the target is exposed to the radiation from 1.0 second to about 10 seconds. In some embodiments, the target is exposed to the radiation from 10 seconds to about 15 seconds. In some embodiments, the target is exposed to the radiation from 15 seconds to 30 seconds. In some embodiments, the target is exposed to the radiation from 30 seconds to 1 minute. In some embodiments, the target is exposed to the radiation from 1 minute to about 5 minutes. In some embodiments, the target is exposed to the radiation from 5 minutes to about 7 minutes. In some embodiments, the target is exposed to the radiation from 7 minutes to about 10 minutes. In some embodiments, the target is exposed to the radiation from 10 minutes to about 20 minutes. In some embodiments, the target is exposed to the radiation from 20 minutes to about 30 minutes. In some embodiments, the target is exposed to the radiation from 30 minutes to about 1 hour. In some embodiments, the target is exposed to the radiation for more than 1 hour.

### Doses, Dose Rates for Tumors

Without wishing to limit the present invention to any theory or mechanism, it is believed that for treating or managing conditions other than macula degeneration (e.g., tumors), a typical dose is expected to be in the range of about 10 Gy to about 100 Gy, such as 85 Gy. Furthermore, it is believed that to irradiate from the exterior side of the eye where the radiation has to pass through the sclera, the RBS should provide a dose rate of about 0.6 Gy/min to about 100 Gy/min to the target. In some embodiments, for treating conditions other than macula degeneration (e.g., tumors), the RBS provides a dose rate of greater than about 10 Gy/min to about 20 Gy/min to the target. In some embodiments, the RBS provides a dose rate of greater than about 20 to 40 Gy/min (e.g., 36 Gy/min) to the target. In some embodiments, the RBS provides a dose rate of greater than about 40 to 60 Gy/min to the target. In some embodiments, the RBS provides a dose rate of greater than about 60 to 80 Gy/min to the target. In some embodiments, the RBS provides a dose rate of greater than about 80 to 100 Gy/min to the target. In some embodiments, the dose rate that is chosen by a user (e.g. physicist, physician) to irradiate the tumor depends on one or more characteristics (e.g., height/thickness of the tumor/lesion (e.g., the thickness of the tumor may dictate what dose rate the user uses).

Without wishing to limit the present invention to any theory or mechanism, it is believed that the exposure time should be from 15 seconds to about 10 minutes for practical reasons. However, other exposure times may be used. In some embodiments, the target is exposed to the radiation from 0.01 seconds to about 0.10 seconds. In some embodiments, the target is exposed to the radiation from 0.10 seconds to about 1.0 second. In some embodiments, the target is exposed to the radiation from 1.0 second to about 10 seconds. In some embodiments, the target is exposed to the radiation from 10 seconds to about 15 seconds. In some embodiments, the target is exposed to the radiation from 15 seconds to 30 seconds. In some embodiments, the target is exposed to the radiation from 30 seconds to 1 minute. In some embodiments, the target is exposed to the radiation from 1 to 5 minutes. In some embodiments, the target is exposed to the radiation from 5 minutes to about 7 minutes. In some embodiments, the target is exposed to the radiation from 7 minutes to about 10 minutes. In some embodiments, the target is exposed to the radiation from 10 minutes to about 20 minutes. In some embodiments, the target is exposed to the radiation from 20 minutes to about 30 minutes. In some embodiments, the target is exposed to the radiation from 30 minutes to about 1 hour. In some embodiments, the target is exposed to the radiation for more than 1 hour.

### Radiation Area, Radiation Profile

In some embodiments, the cannula 100 and/or RBSs of the present invention are designed to treat a small target area with a substantially uniform dose and are also designed so that the radiation dose declines more rapidly as measured laterally from the target as compared to the prior art. The prior art conversely teaches the advantages of a substantially uniform dose over a larger diameter target and with a slower decline in radiation dose (as measured laterally) (e.g., U.S Pat. No. 7,070,544 B2). In some embodiments, the radiation dose rapidly declines as measured laterally from edge of an isodose (e.g., the area directly surrounding the center of the target wherein the radiation dose is substantially uniform).

In some embodiments, the radiation dose at a distance of about 0.5 mm from the center of the target is about 10% less than the dose on the central axis of the target. In some embodiments, the radiation dose at a distance of about 1.0 mm from the center of the target is about 30% less than the dose on the central axis of the target. In some embodiments, the radiation dose at a distance of about 2.0 mm from the center of the target is about 66% less than the dose on the central axis of the target. In some embodiments, the radiation dose at a distance of about 3.0 mm from the center of the target is about 84% less than the dose on the central axis of the target. In some embodiments, the radiation dose at a distance of about 4.0 mm from the center of the target is about 93% less than the dose on the central axis of the target.

In some embodiments, the dose on the central axis of the target is the dose delivered at the choroidal neovascular membrane (CNVM). In some embodiments the radiation dose extends away from the target (e.g., choroidal neovascular membrane) in all directions (e.g., laterally, forwardly), wherein the distance that the radiation dose laterally extends in a substantially uniform manner is up to about 0.75 mm away. In some embodiments the radiation dose extends away from the target in all directions (e.g., laterally, forwardly), wherein the distance that the radiation dose laterally extends in a substantially uniform manner is up to about 1.5 mm away. In some embodiments the radiation dose extends away from the target in all directions (e.g., laterally, forwardly), wherein the distance that the radiation dose laterally extends in a substantially uniform manner is up to about 2.5 mm away.

In some embodiments, the radiation dose at a distance of 2 mm laterally from the center of the target is less than 60% of the radiation dose on the central axis of the target. In some embodiments, the radiation dose at a distance of 3 mm laterally from the center of the target is less than 25% of the radiation dose at the center of the target. In some embodiments, the radiation dose at a distance of 4 mm laterally from the center of the target is less than 10% of the radiation dose at the center of the target. Because the edge of the optic nerve is close to the target, this dose profile provides greater safety for the optic nerve than methods of the prior art.

In some embodiments, the radiation dose is substantially uniform within a distance of up to about 1.0 mm (as measured laterally) from the center of the target. In some embodiments, the radiation dose declines such that at a distance of about 2.0 mm (as measured laterally) from the center of the target, the radiation dose is less than about 25% of the radiation dose at the center of the target. In some embodiments, the radiation dose declines such that at a distance of about 2.5 mm (as measured laterally) from the center of the target, the radiation dose is less than about 10% of the radiation dose at the center of the target.

In some embodiments, the radiation dose is substantially uniform within a distance of up to about 6.0 mm (as measured laterally) from the center of the target. In some embodiments, the radiation dose declines such that at a distance of about 12.0 mm (as measured laterally) from the center of the target, the radiation dose is less than about 25% of the radiation dose at the center of the target. In some embodiments, the radiation dose declines such that at a distance of about 15.0 mm (as measured laterally) from the center of the target, the radiation dose is less than about 10% of the radiation dose at the center of the target.

In some embodiments, the radiation dose is substantially uniform within a distance of up to about 10.0 mm (as measured laterally) from the center of the target. In some embodiments, the radiation dose declines such that at a distance of about 20.0 mm (as measured laterally) from the center of the target, the radiation dose is less than about 25% of the radiation dose at the center of the target. In some embodiments, the radiation dose declines such that at a distance of about 25.0 mm (as measured laterally) from the center of the target, the radiation dose is less than about 10% of the radiation dose at the center of the target.

In some embodiments, the radiation dose at the center of the target (e.g., radiation dose at the center of the choroidal neovascular membrane) does not extend laterally to the entire macula (a diameter of about 1.5 mm to 6.0 mm). In some embodiments, the devices of the present invention may also treat a larger area and still have a faster radiation dose fall off as compared to devices of the prior art.

### Benefit of Short Delivery Time

Without wishing to limit the present invention to any theory or mechanism, it is believed that faster delivery time of radiation is advantageous because it allows the physician to hold the instrument in the desired location with minimal fatigue, and it minimizes the amount of time that the patient is subjected to the procedure. Lower dose rates and longer delivery times may cause fatigue in the physician, possibly leading to the accidental movement of the cannula from the target. Furthermore, longer delivery times increase the chance of any movements of the physician's hand or the patient's eye or head (when local anesthesia is employed, the patient is awake during the procedure).

Another benefit of a faster delivery time is the ability to employ short-term local anesthetics (e.g., lidocaine) and/or systemic induction drugs or sedatives (e.g., methohexital sodium, midazolam). Use of short-term anesthetics result in a quicker recovery of function (e.g., motility, vision) after the procedure. Shorter acting anesthetics cause shorter-lasting respiratory depression in case of accidental central nervous system injection.

The present invention is illustrated herein by example, and various modifications may be made by a person of ordinary skill in the art. For example, although the systems of the present invention have been described above in connection with the preferred sub-Tenon radiation delivery generally above the macula, the systems may be used to deliver radiation directly on the outer surface of the sclera, below the Tenon's capsule, and generally above portions of the retina other than the macula. Moreover, in some embodiments, the systems (e.g., cannulae) of the present invention may be used to deliver radiation from below the conjunctiva and above the Tenon's capsule. In some embodiments, the systems may be used to deliver radiation to the anterior half of the eye. In some embodiments, the systems may be used to deliver radiation from above the conjunctiva. As another example, the arc length and/or radius of curvature of the distal portions of the cannulae may be modified to deliver radiation within the Tenon's capsule or the sclera, generally above the macula or other portions of the retina, if desired.

### Additional Rationale of Device and Methods

Without wishing to limit the present invention to any theory or mechanism, it is believed that the devices of the present invention are advantageous over other posterior radiation devices of the prior art because the devices of the present invention are simpler mechanically and less prone to malfunction. In some embodiments, the devices of the present invention are only used one time.

Without wishing to limit the present invention to any theory or mechanism, it is believed that the unique radiation profile of the present invention is advantageous over the prior art. As discussed previously, the devices and methods of the present invention, which suitably employ the rotationally symmetrical surface concept described above, provide for a more sharply demarcated dose radiation profile from the edge of a substantially uniform dose region. Other posterior devices do not provide this unique radiation profile. The devices and methods of the present invention are advantageous because they will deliver a therapeutic dose of radiation to the target (e.g., neovascular growths affecting the central macula structures) while allowing for the radiation dose to fall off more quickly than the prior art, which helps prevent exposure of the optic nerve and/or the lens to radiation. Further, a faster fall off of the lateral radiation dose minimizes the risk and the extent of radiation retinopathy, retinitis, vasculitis, arterial and/or venous thrombosis, optic neuropathy and possibly hyatrogenic neoplasias.

The present methods of treatment may be used alone or in combination with a pharmaceutical, e.g., for treating Wet Age-Related Macular Degeneration. Non-limiting examples of pharmaceuticals that may be used in combination with the present invention includes a radiation sensitizer an anti-VEGF (vascular endothelial growth factor) drug such as Lucentis® or Avastin®, and/or other synergistic drugs such as steroids, vascular disrupting agent therapies, and other anti-angiogenic therapies both pharmacologic and device-based.

For example, the present invention may be used in combination with a pharmaceutical (e.g., a “first treatment”) such as an anti-angiogenesis treatment for inhibiting an angiogenesis target. The present invention may also be used in combination with more than one pharmaceutical (e.g., a first treatment and a second treatment, etc.). Administration may be simultaneous or consecutive. For example, in some embodiments, the present invention is used in combination with an anti-angiogenesis treatment (e.g., the first treatment) and a steroid treatment (e.g., the second treatment), e.g., corticosteroid treatment.

Without wishing to limit the present invention to any theory or mechanism, non-limiting examples of angiogeneis inhibitors may include angiopoeitin 2, soluble vascular endothelial growth factor receptor-1 (VEGFR-1), soluble Tie2, soluble neuropilin-1 (NRP-1), endostatin, angiostatin, tissue inhibitor of metalloprotease-3 (TIMP-3), prolactin, platelet factor-4, calreticulin, thrombospondin 1 (TSP-1), thrombospondin 2 (TSP-1), Vandetanib (e.g., ZD6474), PDGF inhibitors, pigment epithelium-derived factor (PEDF), VEGF inhibitory aptamers, e.g., Macugen® (pegaptanib, Pfizer), antibodies or fragments thereof, e.g., anti-VEGF antibodies, e.g., bevacizumab (Avastin®, Genentech), or fragments thereof, e.g., ranibizumab (Lucentis®, Genentech), soluble fins-like tyrosine kinase 1 (sFlt1) polypeptides or polynucleotides; PTK/ZK; KRN633; inhibitors of integrins (e.g., αvβ3 and a5βI); VEGF-Trap® (Regeneron); Alpha2-antiplasmin, cartilage-derived angiogenesis inhibitor (CDAI), disintegrin and metalloproteinase with thrombospondin motifs 1 (ADAMTS1), ADAMTS2, IFN-alpha, IFN-beta, IFN-gamma, chemokin (C—X—C motif) ligand 10 (CYCL10), IL-4, IL-12, IL-18, prothrombin, antithrombin III fragments, and vascular endothelial growth inhibitor (VEGI), anti-hypoxia inducible factor-1 (anti-HIF-1) compounds or treatments, and immunosuppressant compounds such as calcineurin inhibitors (e.g., tacrolimus) or mTOR inhibitors (e.g., rapamycin), e.g., see W.O. Pat. Application No. 2008/119500, the disclosure of which is incorporated in its entirety by reference herein.

Without wishing to limit the present invention to any theory or mechanism, other angiogenesis inhibitors may also be found in U.S. Patent Application No. 2008/0193499; U.S. Pat. No. 7,091,175, U.S. Patent Application 2001/0041744; U.S. Pat. No. 6,472,379; U.S. Pat. Application No. 2002/0001630; U.S. Patent Application 2005/0043220; U.S. Patent Application 2004/0048807, the disclosures of which re incorporated in their entirety by reference herein.

Without wishing to limit the present invention to any theory or mechanism, non limiting examples of corticosteroids are I′-alpha, 17-alpha, 21-trihydroxypregn-4-ene-3,20-dione; 11-beta, 16-alpha, 17, 21-tetrahydroxypregn-4-ene-3, 20-dione; 11-beta, 16-alpha, 17, 21-tetrahydroxypregn-I, 4-diene-3, 20-dione; 11-beta, 17-alpha, 21-trihydroxy-6-alpha-methylpregn-4-ene-3,20-dione; 11-dehydrocorticosterone; 11-deoxycortisol; 11-hydroxy-I, 4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-I, 4,9(11)-triene-3, 20-dione; 17-alpha-hydroxypregn-4-ene-3, 20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-di-ene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha, 20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; Cortisol; Cortisol acetate; Cortisol butyrate; Cortisol cypionate; Cortisol octanoate; Cortisol sodium phosphate; Cortisol sodium succinate; Cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone bpteprate; hydrocortisone butyrate; hydrocortisone cypionate; hydrocortisone hem isuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hem isuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

The terms “compound that inhibits VEGF,” “anti-VEGF treatment,” and/or “anti-VEGF drug” may refer to a compound (or the like) that inhibits the activity or production of vascular endothelial growth factor (VEGF). For example, the terms may refer to compounds capable of binding VEGF, including small organic molecules, antibodies or antibody fragments specific to VEGF, peptides, cyclic peptides, nucleic acids, antisense nucleic acids, RNAi, and ribozymes that inhibit VEGF expression at the nucleic acid level. Non-limiting examples of compounds that inhibit VEGF are nucleic acid ligands of VEGF (e.g., such as those described in U.S. Pat. No. 6,168,778 or U.S. Pat. No. 6,147,204), EYEOOI (previously referred to as NX1838) which is a modified pegylated aptamer that binds with high affinity to the major soluble human VEGF isoform; VEGF polypeptides (e.g., U.S. Pat. No. 6,270,933 and WO Pat. No. 99/47677); oligonucleotides that inhibit VEGF expression at the nucleic acid level, for example antisense RNAs (e.g. U.S. Pat. No. 5,710,136; U.S. Pat. No. 5,661,135; U.S. Pat. No. 5,641,756; U.S. Pat. No. 5,639,872; U.S. Pat. No. 5,639,736). Other examples of inhibitors of VEGF signaling known in the art may include, e.g., ZD6474; COX-2, Tie2 receptor, angiopoietin, and neuropilin inhibitors; PEDF, endostatin, and angiostatin, soluble fins-like tyrosine kinase 1 (sFltl) polypeptides or polynucleotides; PTK787/ZK222 584; KRN633; VEGF-Trap® (Regeneron); and Alpha2-antiplasmin. Compounds that inhibit VEGF may be antibodies to, or antibody fragments thereof, or aptamers of VEGF or a related family member such as (VEGF B. I C, D; PDGF). Examples are anti-VEGF antibodies, e.g., Avastin™ (also reviewed as bevacizumab, Genentech), or fragments thereof, e.g., Lucentis™ (also reviewed as rhuFAb V2 or AMD-Fab; ranibizumab, Genentech), and other anti-VEGF compounds such as VEGF inhibitory aptamers, e.g., Macugen™ (also reviewed as pegaptanib sodium, anti-VEGF aptamer or EYEOOI, Pfizer). In some embodiments, a compound that inhibits VEGF can further be an immunosuppressant compound (e.g., calcineurin inhibitors, mTOR inhibitors). The disclosures of all referenced U.S. Patents and W.0 Patents are incorporated in their entirety by reference herein.

In some embodiments, the pharmaceutical (e.g., angiogenesis inhibitor, e.g., VEGF-inhibitor, etc.) may comprise a compound selected from the group consisting of an oestrogen (e.g. oestrodiol), an androgen (e.g. testosterone) retinoic acid derivatives (e. g. 9-cis-retinoic acid, 13-trans-retinoic acid, all-trans retinoic acid), a vitamin D derivative (e. g. calcipotriol, calcipotriene), a non-steroidal antiinflammatory agent, a selective serotonin reuptake inhibitor (SSRI; e.g. fluoxetine, sertraline, paroxetine), a tricyclic antidepressant (TCA; e.g. maprotiline, amoxapine), a phenoxy phenol (e.g. triclosan), an antihistaminine (e.g. loratadine, epinastine), a phosphodiesterase inhibitor (e.g. ibudilast), an anti-infective agent, a protein kinase C inhibitor, a MAP kinase inhibitor, an anti-apoptotic agent, a growth factor, a nutrient vitamin, an unsaturated fatty acid, and/or ocular anti-infective agents, for the treatment of the ophthalmic disorders set forth herein (see for example compounds disclosed in US 2003/0119786; WO 2004/073614; WO 2005/051293; US 2004/0220153; WO 2005/027839; WO 2005/037203; WO 03/0060026). Anti-infective agents may include but are not limited to penicillins (ampicillin, aziocillin, carbenicillin, dicloxacillin, methicillin, nafcillin, oxacillin, penicillin G, piperacillin, and ticarcillin), cephalosporins (cefamandole, cefazolin, cefotaxime, cefsulodin, ceftazidime, ceftriaxone, cephalothin, and moxalactam), aminoglycosides (am ikacin, gentamicin, netilmicin, tobramycin, and neomycin), miscellaneous agents such as aztreonam, bacitracin, ciprofloxacin, clindamycin, chloramphenicol, cotrimoxazole, fusidic acid, imipenem, metronidazole, teicoplanin, and vancomycin), antifungals (amphotericin B, clotrimazole, econazole, fluconazole, flucytosine, itraconazole, ketoconazole, miconazole, natamycin, oxiconazole, and terconazole), antivirals (acyclovir, ethyldeoxyuridine, foscarnet, ganciclovir, idoxuridine, trifluridine, vidarabine, and (S)-1-(3-dydroxy-2-phospho-nyluethoxypropyl) cytosine (HPMPC)), antineoplastic agents (cell cycle (phase) nonspecific agents such as alkylating agents (chlorambucil, cyclophosphamide, mechlorethamine, melphalan, and busulfan), anthracycline antibiotics (doxorubicin, daunomycin, and dactinomycin), cisplatin, and nitrosoureas), antimetabolites such as antipyrimidines (cytarabine, fluorouracil and azacytidine), antifolates (methotrexate), antipurines (mercaptopurine and thioguanine), bleomycin, vinca alkaloids (vincrisine and vinblastine), podophylotoxins (etoposide (VP-16)), and nitrosoureas (carmustine, (BCNU)), and inhibitors of proteolytic enzymes such as plasminogen activator inhibitors.

The pharmaceutical (or combination of pharmaceuticals) may include an anti-paracyte (e.g., a paracyte is a cell that adheres and matures new vessels). The pharmaceutical (or combination of pharmaceuticals) may include a VEGF-Trap (Bayer). The pharmaceutical (or combination of pharmaceuticals) may comprise a PDT agent, e.g., verteporfin (Visudyne, Novartis), SnET2 PDT (Photrex, Miravant); a steroid, e.g., anecoratve (Retaane Depot, Alcon), dexamethasone (Posurdex, Allergan), floucinolone acetonide (Iluvien, Alimera Sciences/pSivida); a microtubule de-stabilizer/VDA, e.g., CA4P (Zybresta, OXiGENE); a PEGylated anti-VEGF aptamer, e.g., pegaptanib (Macugen, OSI/Pfizer/Gilead Sciences); a Pan-VEGF Fab′, e.g., ranulizumab (Lucentis, Genentech/Novartis); bevacizumab (Avastin, Genentech); a small molecule VEGFR inhibitor, e.g., AG-13958 (Pfizer); VEGF-Trap (Regeneron/Bayer); a soluble decoy fusion protein comprising portions of VEGFR1 and VEGFR2 that binds VEGF and PIGF, e.g., AVE0005 (Aflibercept (Regeneron/Sanofi); an RNAi, e.g., Sirna-027 (Merck/Allergan), ALN-VEG01 (Alnylam); an anti-a5B1 integrin Fab′, e.g., F200 (Protein Design Lab); ACZ885F (Novartis); an anti-angiogenic compound, e.g., ATG-3/ATG-003 (CoMentis), an NSAID, e.g., xibrom bromfenac (Ista Pharmaceuticals Inc & Senju Pharmaceutical Co.); a small molecule resolvin, e.g., NPDI (Resolvyx); RX 10001 (RvEI); RX 10008; an immunosuppressive, anti-angiogenic, anti-migratory, anti-proliferative, anti-fibrotic, and/or anti-permeability compound, e.g., sirolimus (e.g., rapamycin) DE-109 (Santen & MacuSight); a siRNA, e.g., bevasiranib (OPKO Health Inc); an antibody fragment, e.g., ESBA903 (ESBATech); a complement inhibitor, e.g., POT-4 (Potentia); a fusion protein (consisting of a complement receptor 2 (CR2) fragment linked to an inhibitory domain of complement factor H (CFH) that inhibits the complement alternative pathway), e.g., TT30 (Taligen); an alpha(5)beta(1) integrin, e.g., JSM-6427; a monoclonal antibody (against sphingosine 1-phosphate), e,g., sphingomab (Lpath Inc.); squalamine; an anti-PDGF aptamer against PDGF-B, e.g., E10030 (Ophthotech); an anti-C5 aptamer, e.g., ARC1905 (Ophthotech); a stem cell based therapy, e.g., HuCNS-SC (StemCells Inc); pyrimidine derivatives, benzodiazepinyl derivatives; embryonic stem cell therapies; aminoacyl tRNA synthetase fragments; PDGF anti-cancer drugs in combination with Lucentis (e.g., Gleeve tablet+Lucentis); amino acid peptide isolated from scorpion venom (e.g., TM601, chlorotoxin, TransMolecular Inc); RetinoStat (Oxford BioScience/Sanofi Aventis); RetinoStat (Oxford BioMedica); adPEDF (GenVec); adenoviral PEDG gene therapy; protein therapeutics, molecular diagnostics; stem cells; non-steroidal inhibior of CREB regulated transcription coactivator-1 and TORC, e.g., Palomid 529 (P529) (Paloma Pharmaceuticals); antibodies; Isonep; sustained release Lucentis; Macroesis (Buckeye); immunoconjugates (e.g., hl-con1, Iconic Therapeutics); heterocomplex of VEGG linked to a keyhole limpet hemocyanin (e.g., VEGF Kinoid, Neovacs); hESCs; microplasmin, e.g., MIVIS (ThromboGenics); compound regulating lipase gene; Medidur FA (pSivida/Alimera); a small interfering RNA product, e.g., RTP-801i-14 (Pfizer/Quank/Silence Therapeutic); a siRNA targeting hypoxia-inducible genes, e.g, RTP801 (Pfizer/Quark); PTK787; ACE-041 (Acceleron Pharma); rC3-1 (InaCode BioPharmaceutics, Inc.); ACU-4429 (Acucela); ACU-02 (Acucela); a covalently-linked form of zinc monocysteine complex, e.g., Zinthionein (Pipex, Adeona Pharmaceuticals); I-vation (SurModics Inc/Merck); phosphatase-activated tumor vascular targeting agent, e.g., Zybrestat (Oxigene); retinylamine (Case Western University); TRC093 TRC 105 (Tracon Pharmaceuticals); volociximab (Opthotech); E10030 (Ophthotech); SAR 1118 (SARcode Corp); Intelligent Retinal Implant System; RNAi drugs (Traversa, for example); PMX53 (Arana Therapeutics); SMT D004 (Summit Corpic); aptamer therapeutics (Archemix Corp); MC1101 (MacuClear); infra-red light; iCo-007 (iCo Therapeutics); NT-501 (Neurotech Pharmaceuticals); OT-551 (Othera Pharmaceuticals); VPC51299 (Catena Pharmaceuticals); fenretinide (Sirion Therapeutics); an anti-angiogenic and/or angiolytic compount, e.g., Eye Drop (OcuCure Therapeutics); CCR3 Marker (University of Kentucky); Alterase (Medlmmune/Calaylst Biosciences); iPS (Japan Kyoto University); CLT-003 (Charlesson); OXY111A (NormOxys Inc.); a photosensitizer (e.g., Photrex, Miravant Medical Technologies); Visudyne with Triamcinolone (QLT); Bevasiranib with ranibizumab (Opko Health); VEGF Trap-Eye (Bayer and Regeneron); AGN745 (Allergan and Merck & Co.); Visudyne with Lucentis, Dexamethasone (QLT); Medidur FA with Lucentis (pSivida); Lucentis with Visudyne (Genentech); TG100801 (Targeted Genetics); ST602 (Sirion Therapeutics) Neurosolve (Vitreoretinal Technologies); an NF-kB inhibitor (e.g., OT551, Othera Pharmaceuticals); DNA-damage-inducible transcript 4 (DDIT4) inhibitor (e.g., RTP801i, Quark Pharmaceuticals and Silence Therapeutics); a C-Kit Receptor Tyrosine Kinase (CD117) Inhibitor (e.g., Armala, GlaxoSmithKline); a Ciliary Neutrophic Growth Factor (CNTF) Receptor Agonist (e.g., NT501, Neurotech); an Alpha7 Non-Neuronal Nicotinic Receptor Antagonist (e.g., ATG003, CoMentis); a slow-released corticosteroid (e.g., Iluvien, Alimera); a Sphingosine-1-Phosphate (S1P) Receptor Antagonist (e.g., Isonep, Lpath); a polyamine analogue (e.g., PG11047, Progen Pharmaceuticals); E10030 with Lucentis (Ophthotech); pigment epithelial derived GF, e.g., AdPEDF (GenVec); an mTOR inhibitor, e.g., Sirolimus (MacuSight); a Non-retinoid visual cycle modulator, e.g., ACU4429 (Otsuka Pharmaceutical/Acucela); a C5 Complement Inhibitor/VEGF-A Inhibitor, e.g., ARC1905 with Lucentis (Ophthotech); an antisense insulin receptor Substrate 1 (IRS-1), e.g., GS101 (Gene Signal); an alpha(5)beta(1) integrin receptor agonist, e.g., SB267268 (GlaxoSmithKline), Volociximab (Biogen Idec and PDL BioPharma); JSM6427 (Jerini/PR Pharmaceuticals).

The pharmaceutical (or combination of pharmaceuticals) may further comprise a pharmaceutically acceptable carrier. Such pharmaceutical carriers are well known to one of ordinary skill in the art. In some embodiments, the pharmaceutical (or combination of pharmaceuticals) may be administered as a slow-release formulation. Such slow-release formulations are well known to one of ordinary skill in the art, for example the pharmaceutical may be in the form of a vehicle such as a micro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers such as those disclosed in U.S. Pat. Nos. 6,667,371; 6,613,355; 6,596,296; 6,413,536; 5,968,543; 4,079, 038; 4,093,709; 4,131,648; 4,138,344; 4,180,646; 4,304,767; 4,946,931, or lipids that may be formulated as microspheres or liposomes. The formulation and loading of microspheres, microcapsules, liposomes, etc. are standard techniques known by one skilled in the art.

Cannulas for Administration of Pharmaceuticals in Combination with RBS

The system (100) may deliver both the radiation from the RBS (180) and at least one pharmaceutical (e.g., as described above). Administration may be simultaneous or consecutive. The pharmaceuticals may be housed in a chamber (or multiple chambers) disposed in the cannula, for example in the proximal portion (130), in the handle (140), in the distal portion (120), in the RBS holder (230), etc. In some embodiments, the system (100) (e.g., the cannula (105), e.g., distal portion (110), RBS holder (230), etc.) comprises a first chamber for housing a first pharmaceutical. In some embodiments, the system (100) (e.g., the cannula (105), e.g., distal portion (110), RBS holder (230), etc.) comprises a first chamber for housing a first pharmaceutical and a second chamber for housing a second pharmaceutical. In some embodiments, the system (100) (e.g., the cannula (105), e.g., distal portion (110), RBS holder (230), etc.) comprises a first chamber for housing a first pharmaceutical, a second chamber for housing a second pharmaceutical, and a third chamber for housing a third pharmaceutical.

In some embodiments, the chamber(s) in the system (100) (e.g., the cannula (105), e.g., distal portion (110), RBS holder (230), etc.) are pre-loaded with a fixed volume of the pharmaceutical(s). In some embodiments, the chamber(s) comprise an opening for providing access to the chamber(s), for example for loading the chamber(s) with the pharmaceutical(s). A closing mechanism may be disposed on the opening, wherein the closing mechanism can move between an open position and a closed position for respectively allowing and preventing access to the chamber (e.g., preventing leaking of the pharmaceutical).

The system (100), e.g., the cannula (105), may comprise one or more pharmaceutical orifices for dispensing the pharmaceutical. The pharmaceutical orifice may be disposed on the distal portion (110), for example at or near the tip of the distal portion (110), e.g., on or in the RBS holder (230). The pharmaceutical orifice may be disposed adjacent to the treatment position of the RBS (180).

The chamber(s) housing the pharmaceutical(s) are fluidly connected to one or more pharmaceutical orifices. For example, in some embodiments, the first chamber is fluidly connected to a first pharmaceutical orifice. In some embodiments, the first chamber is fluidly connected to a first pharmaceutical orifice and the second chamber is also fluidly connected to the first pharmaceutical orifice. In some embodiments, the first chamber is fluidly connected to a first pharmaceutical orifice and the second chamber is fluidly connected to a second pharmaceutical orifice. The chamber(s) housing the pharmaceutical(s) may be fluidly connected to the pharmaceutical orifice via a tube.

In some embodiments, the system (100), e.g., cannula (105), comprises one or more advancing mechanisms for advancing the pharmaceutical from the chamber(s) to the pharmaceutical orifice(s), e.g. via a tube, and ultimately causing the dispensing of the pharmaceutical. Non-limiting examples of such mechanisms include pneumatic mechanism (e.g., air pressure), a vacuum mechanism, a slide mechanism, a button mechanism, a dial mechanism, a thumb ring, a graduated dial, a slider, a fitting, a Toughy-Burst type fitting, a plunger (e.g., a solid stick, a piston, a shaft, etc.), a hydrostatic pressure mechanism, the like, or a combination thereof. For example, a plunger mechanism may push the pharmaceutical (e.g., angiogenesis inhibitor) from the chamber to the orifice via the tube. The pharmaceutical orifices may be constructed in a variety of shapes, for example shapes such as a circle, a square, an oval, a rectangle, an ellipse, a triangle, or an irregular shape.

The device of the present invention may be constructed from a variety of materials. For example, in some embodiments, the device is constructed from a material comprising polyetherimide material (e.g., Ultem®), plastic, glass, (e.g., Gorilla® Glass), acrylic, poly(methyl methacrylate) (PMMA), polysulfone, polycarbonate, polypropylene, stainless steel, aluminum, titanium, elgiloy, lead, the like, or a combination thereof. The present invention is not limited to the aforementioned materials.

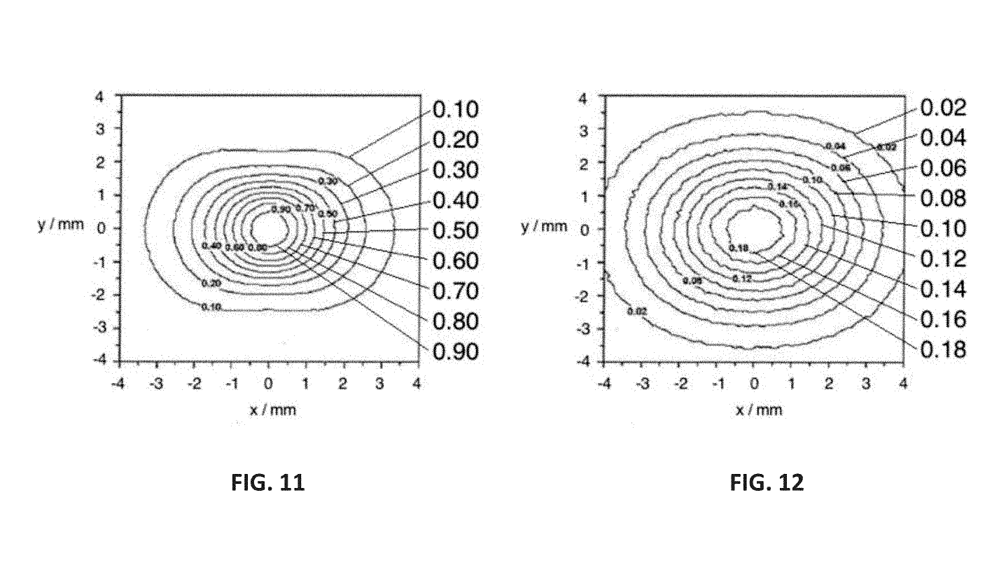

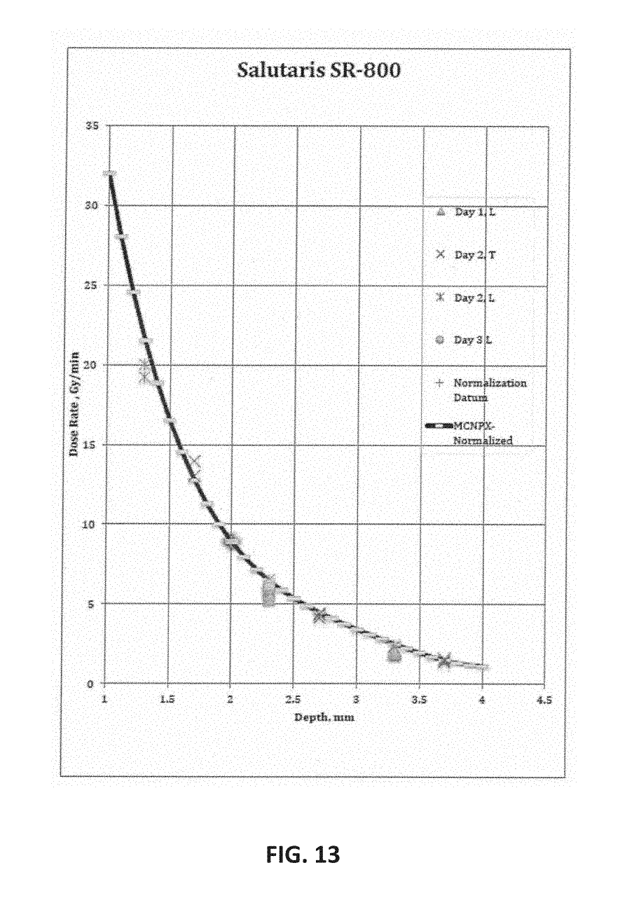

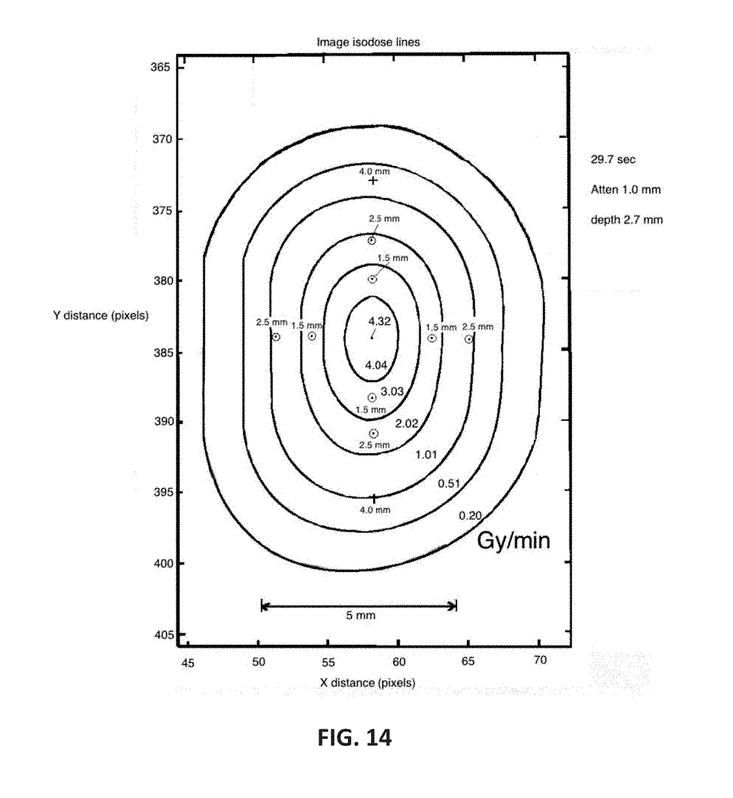

For reference, FIG. 10 is a detailed view of an example of a radionuclide brachytherapy source (RBS) (or radiation source) at the tip of the cannula of the device of the present invention. FIG. 11 is a diagram showing the relative dose distribution in Gy/min at 1.5 mm distance from the source. The isodose lines are normalized to the central point. FIG. 12 is a diagram showing the relative dose distribution in Gy/min at 3 mm distance from the source. The isodose lines are normalized to the central point at 1.5 mm depth. FIG. 13 is a diagram showing the several central axis dose determinations on different days and the two different techniques for setting the depths. The dose rate is shown as a function of depth from the source center for the several measurements conducted, the one determination at 2.0 by the manufacturer, and the MCNPX calculations normalized to 8.9 Gy/min at 2.0 mm. Experimental dose rates were determined from the dose measured from an exposure divided by the time of the exposure. The label “T” refers to measurement in the treatment configuration (cylindrical phantom) while “L” refers to the lateral measurement using the side of the cannula on the flat phantom. FIG. 14 is a diagram showing isodose lines at 2.7 mm determined experimentally and analyzed by hand. The dose rates are determined from the absolute doses measured for this exposure divided by the time of the exposure.

### EXAMPLE 1

### Surgical Technique

The following example describes a surgical procedure for use of the cannulae of the present invention. The eye is anesthetized with a peribulbar or retrobulbar injection of a short acting anesthetic (e.g., Lydocaine). A button hole incision in the superotemporal conjunctiva is preformed followed by a button hole incision of the underlying Tenon capsule.

A small conjunctive peritomy (as large as the diameter of the distal chamber) is performed at the superotemporal quadrant. A Tenon incision of the same size is then performed in the same area to access the subtenon space.

Balanced salt solution and/or lydocaine is then injected in the subtenon space to separate gently the Tenon capsule from the sclera.

The distal portion (110) of the cannula is then inserted in the subtenon space and slid back until the RBS holder (230) (with the RBS (180) therein) is at the posterior pole of the eye. In some embodiments, the surgeon adjusts the position of the cannula (105) while the patient's eye is in a primary gaze position. In some embodiments, the surgeon adjusts the position of the cannula (105) while the patient's eye is in any one of the following position: elevated, depressed, adducted, elevated and adducted, elevated and abducted, depressed and adducted, and depressed and abducted.

In some embodiments, the system (100) (e.g., cannula (105)) comprises a light system, e.g., a light emitting component (150) disposed on or in the RBS holder (230). The light may be seen through transillumination and may help guide the surgeon to the correct positioning of the system (100) (e.g., cannula (105)). In some embodiments, the light is directed from a light source through the system (100) by fiberoptics. In some embodiments, the light system comprises a LED.

The system with the RBS is left in place for the desired length of time. When the planned treatment time has elapsed, the system (100) (e.g., distal portion (110) of the cannula) may then be removed from the subtenon space. The conjunctiva may then be simply reapproximated or closed with bipolar cautery or with one, two, or more interrupted reabsorbable sutures.

The button hole cunjunctiva/tenon incision has several advantages over a true conjunctiva/Tenon incision. It is less invasive, faster, easier to close, more likely to be amenable to simple reapproximation, less likely to require sutures, and causes less conjunctiva scarring (which may be important if the patient has had or will have glaucoma surgery).

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. The reference numbers recited in the below claims are solely for ease of examination of this patent application, and are exemplary, and are not intended in any way to limit the scope of the claims to the particular features having the corresponding reference numbers in the drawings. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A brachytherapy applicator system (100) comprising:
a. a cannula (105) comprising: a curved distal portion (110) for placement around a portion of a globe of an eye, the distal portion (110) has a radius of curvature from 9 to 15 mm and an arc length from 25 to 35 mm; and a curved proximal portion (120) connected to the distal portion (110) by an inflection point (130) or a straight portion (132), the proximal portion (120) has a radius of curvature from 1 mm to 500 mm; and
b. a radionuclide brachytherapy source (RBS) holder (230) directly or indirectly connected to the distal portion (110), the RBS holder (230) comprises a cavity adapted to hold a radionuclide brachytherapy source (RBS) (180) and a cap (248) removably attachable to the RBS holder (230) for sealing the cavity (248), wherein the cap (248) comprises a head configuration (249) to allow for engagement with a tool that can move the cap (248) in a manner so as to secure the cap (248) to the RBS holder (230) and seal the cavity (232);
wherein an angle θ₁ between (i) a line l₃ tangent to the inflection point (130) or straight portion (132) and (ii) the proximal portion (120) is from greater than 0 degrees to 180 degrees.

2. The system (100) of claim 1, wherein said cannula (105) is for placing under a Tenon's capsule.

3. The system (100) of claim 1 further comprising a handle (140) connected to the proximal portion (120).

4. The system (100) of claim 3, wherein the handle (140) is connected to the proximal portion (120) via a straight proximal portion (134).

5. The system (100) of claim 3, wherein the handle (140) lies on an axis such that the axis does not intersect with the distal portion (110).

6. The system of claim 3, wherein the handle (140) comprises a gripping component (144) for helping a user hold the handle (140).

7. The system of claim 6, wherein the gripping component (144) comprises grooves, bumps, indentations, or scratches.

8. The system of claim 3, wherein the handle (140) further comprises an alignment component (143) for providing a user a visual or tactile marking for determining orientation of the distal portion (110) or RBS holder (230).

9. The system of claim 8, wherein the alignment component (143) comprises a visual mark or visual distinction.

10. The system (100) of claim 8, wherein the alignment component (143) comprises an indentation, a bump, or a ridge.

11. The system of claim 8, wherein the alignment component is disposed on or in a distal portion of the handle (140).

12. The system of claim 1, wherein the head configuration (249) comprises at least one indentation or slot in a top surface of the cap (248).

13. The system of claim 12, wherein the head configuration (249) comprises a single slot, a pair of slots, a pair of indentations, a cruciform shaped screw drive or an internal hex.

14. The system of claim 1 wherein the head configuration comprises at least one external side edge different from other external side edges.

15. The system of claim 14, wherein the head configuration (249) comprises an external hex.

16. The system of claim 1, wherein the cap (248) is a snap-on cap adapted to fit onto the RBS holder (230) or within the RBS holder (230).

17. The system (100) of claim 1, wherein the RBS holder (230) is connected to the distal portion (110) via a straight distal portion (136).

18. The system (100) of claim 1, wherein the RBS holder (230) is connected to the distal portion (110) via a kink (138).

19. The system (100) of claim 1, wherein the RBS holder (230) is connected to the distal portion (110) via a kink (138) and a straight distal portion (136), wherein the kink (138) is connected to the distal portion (110) and the straight distal portion (136) is connected to the RBS holder (230).

20. The system of claim 17, wherein the straight distal portion (136) engages a socket (231) in the RBS holder (230).

21. The system of claim 18, wherein the kink (138) engages a socket (231) in the RBS holder (230).

22. The system of claim 19, wherein the straight distal portion (136) engages a socket (231) in the RBS holder (230).

23. The system of claim 17, wherein the straight distal portion (136) has a length from 0.1 mm to 25 mm.

24. The system of claim 18, wherein the kink (138) has a radius of curvature from 1 to 500 mm.

25. The system of claim 18, wherein the kink (138) has an arc length from 0.1 to 20 mm.

26. The system of any of claims 1 further comprising a light system (150) adapted to emit light from the RBS holder (230) or distal portion (110).

27. The system of claim 26, wherein the light system (150) comprises a light emitting diode (LED) disposed in the RBS holder (230).

28. The system of claim 26, wherein the light system (150) comprises a fiber optic light wire (152) disposed on at least a bottom surface of the RBS holder (230), wherein light is emitted from a tip of the fiber optic light wire (152).

29. The system of claim 27, wherein the fiber optic light wire (152) extends through the distal portion (110) and proximal portion (120).

30. The system (100) of claim 1, wherein an RBS is loaded into the RBS holder (230) prior to insertion of the system (100) in a patient.
